(19) United States
(12) Patent Application Publication
(10) Pub. No.: US 2011/0112573 A1
(43) Pub. Date: May 12, 2011

(54) METHODS AND DEVICES FOR USE WITH SEALANTS

(76) Inventor: Orahn Preiss Bloom, Zichron Yakov (IL)

(21) Appl. No.: 13/000,019

(22) PCT Filed: Jun. 18, 2009
(86) PCT No.: PCT/IB09/52600

§ 371 Date: Dec. 20, 2010

Related U.S. Application Data
(60) Provisional application No. 61/129,320, filed on Jun. 18, 2008.

(51) Int. Cl. A61B 17/00

(52) U.S. Cl. 606/213

(57) ABSTRACT

A biocompatible medical sealant for use in a biological system, the sealant comprising a solution of a cross-linkable protein or polypeptide and a solution of a non-toxic cross-linking material which induces cross-linking of said cross-linkable protein, thereby sealing at least a portion of the biological system.

### FIELD OF THE INVENTION

The present invention relates to biocompatible medical sealants, and to uses thereof in biological and physiological systems.

### BACKGROUND OF THE INVENTION

The living human organism contains pressurized fluids, such as blood, urine, lymph, bile, cerebral spinal fluid (CSF), intestinal fluid and air. The liquids are contained in a closed system of vessels, while air is pressurized in the alveolus of the lungs during the inhalation part of the breathing cycle.

The liquid-containing vessel systems can be divided into two categories, high pressure systems and low pressure systems. The arterial blood vessels have the highest pressure, with pulsating pressure in the range of 70-140 mmHG in healthy humans, reaching as high as 220 mmHg in patients suffering from cardiovascular hypertension. Major veins such as the vena cava also show high pulsating pressure, but not as high as that of the arteries. Low pressure systems (having pressure in the range of 10-60 mmHG) include the urinary tract, and systems containing lymph, bile, CSF and intestinal intraluminal content within the gastro intestinal.

Damage to the liquid-containing vessels may occur as a result of surgery, trauma or disease, resulting in leakage of the liquid. Repair of damaged vessels is currently achieved by use of sutures and staples.

Typically, a surgical stapler comprises two stapler arms, one containing one or more lines of multiple staples and a second containing a corresponding structure to bend each of the staples into a closed position. A wide array of stapling devices from different manufacturers is currently available. These vary in staple size, gap width, and staple shape, each having its inherent drawbacks.

The use of stapler devices may result in the leakage of body fluids, such as gastro intestinal content, urine, bile or cerbro spinal fluid (CSF), and in the lungs it can cause pneumothorax.

For some procedures, the use of bare staples, with the staples in direct contact with the patient's tissue, is generally acceptable. The integrity of the tissue itself will normally prevent the staples from tearing out of the tissue and compromising the seam before healing has occurred. In certain circumstances, however, the tissue that is being sealed is too fragile to securely hold the staples in place. In these instances, the tissue will tend to rip at or near the staple lines, slowing healing and often leading to serious complications.

One area where fragile tissue is of particular concern is the use of stapler devices in lung tissue, and especially lung tissue that is affected by emphysema or similar condition. Diseased lung tissue is very fragile and, in extreme cases, will readily tear through unprotected staple lines. With the growing use of surgical staplers in operations on diseased lung tissues such as bullectomies and volume reduction procedures, it has become increasingly important to develop some reliable means to protect fragile tissue from tissue tears due to surgical staples or surgical stapling procedures. Moreover, when staples are used, it is desirable to reduce any leakage around the staples.

Other staplers such as endoscopic staplers present other difficulties. An endoscopic stapler is constructed to allow the stapler to be inserted through a small incision and then operated remotely within a patient's body by the surgeon. To accomplish this, most endoscopic staplers comprise shorter stapler arms (or “jaws”) that are connected together on a fixed pivot point in a scissors fashion. The stapler arms are generally mounted remotely from the surgeon's actuation means through an extended staff.

Use of endoscopic staplers presents a number of unique problems. First, it has been found that the scissors-like construction of the stapler arms tends to entrap tissue within the pivot point. This can cause fouling problems within the pivot point. Additionally, the remote nature of the endoscopic stapler can make removal of excess reinforcement material difficult from the surgical site. Finally, secure retention of reinforcement material on remote arms is a major concern for a surgeon.

For many applications, a surgical blade is included in the device to quickly sever tissue between the lines of staples. These allow for quick division and closure of tissue, which shortens the operating time. Such devices are suitable for use with most types of tissue. In abdominal surgery, linear and circular cutting/stapling devices have been commonly used for many years. For minimally invasive surgery, devices adapted to pass through a trocar are available.

Those stapler devices employing a cutting blade are referred to as “anastomotic staplers” and those used without a cutting blade are referred to as “non-anastomotic staplers.”

In the operation of a typical anastomotic stapler, the two stapler arms are positioned around tissue to be cut and then locked firmly together. In one motion, the user actuates the stapler device, which simultaneously installs two or more lines of staples through the tissue and cuts a line down the middle of the staple lines. In this manner, the user can quickly cut and seal tissue at the same time. This procedure is much faster than using a conventional process of cutting with scissors or a scalpel and then laboriously sealing the incision with sutures. As a result, patient care is dramatically improved by minimizing bleed time from the surgical site and significantly increasing the speed with which an operation can be completed.

Stapled resection and anastomosis have not shown fewer complications than hand-sewn procedures. However, their use has become standard in many operations, because of the shortened operating time and reduced tissue manipulation.

The two main causes of mortality, major complications and enormous costs of gastric bypass, bilio-pancreatic diversions, etc., are the improper healing of the anastomosis, called dehiscence (a premature bursting open or splitting along a surgical suture/staple line, such as in the junction or connection between the ends of the intestine, or the stomach pouch and the intestine) and pulmonary thromboembolism. The mortality caused by anastomotic dehiscence ranges from 30 to 50%.

In particular, in gastro intestinal anastomoses, it was reported that in 5-15% of cases, leakage occurs from the suture/staple line (source: Colorectal Surgery and Anastomotic Leakage; P. B. Soetersa, J. P. J. G. M. de Zoetea, C. H. C. Dejonga, N. S. Williamsb, C. G. M. I. Baetena; Dig Surg 2002;19:150-155). The leaks of contaminated fluids often result in peritonitis, re-intervention and the need for protective stoma. Higher leak rate and higher morbidity and mortality are specifically apparent in patients with problematic nutritional states, chronic inflammation (Crohn's disease, Colitis) or liver disease.

Especially for colonic anastomosis, where leakage has the most devastating consequences, many attempts have been made to decrease the problem of anastomotic dehiscence by reinforcing the anastomosis and to facilitate construction.

It is known to use bovine pericardial tissue as a staple line reinforcement sleeve. During an operation, a surgeon staples and cuts through both the bovine pericardial tissue and the patient's tissue. Once the staples are in place, the surgeon must then cut the suture lines holding the bovine pericardial strips in place and remove the polyethylene backing material and sutures.

In the past years, products in the form of implantable strips that are intended to commercialize or replace bovine pericardium strips have been disclosed. Such products are described, for example, in U.S. Patent Applications No. 20040093029, 20030120284, and 20070246505, as well as U.S. Pat. Nos. 5,752,965 and 5,810,855.

A surgical stapler reinforcement material is disclosed in U.S. Pat. No. 5,441,193 to Gravner, wherein a resilient strip of material is pre-attached to a stapler jaw and/or anvil. The surgical staples are fired and set through the tissue and resilient material which strengthens and reinforces the staples. The resilient material can be pre-attached to the stapler by the use of adhesives or by mechanical means such as grooves, slots or projections. Once the staples are fired, the reinforcement material is released from the stapler jaw and/or anvil. Since the reinforcement material of Gravner is pre-attached to the stapler, it is only suited for those staplers specifically designed to receive such a configuration. Due to the integral nature of the stapler and the reinforcement material, no carrier facilitating the loading of the reinforcement material onto the stapler is required.

In U.S. Pat. Nos. 5,503,638, 5,575,803 and 5,549,628 to Cooper et al., an alternate configuration of a staple reinforcement material is disclosed, wherein a disposable sleeve is attached to the reinforcement material. The sleeve is formed into a three-sided “U” shape, which is sized to slip-fit over a stapler jaw or anvil. The fourth side of the sleeve is comprised of the reinforcement material which contacts the active surface of stapler jaw or anvil. The reinforcement material is releasably attached to the disposable sleeve, for example by a suture. After the staples are fired, the reinforcement material is released from the disposable sleeve by unthreading the suture. The disposable sleeve must then be removed and discarded. Such a reinforcement material is more suited for open surgical procedures. In laparoscopic procedures, the sleeve surrounding the stapler jaw and anvil can interfere with the trocar. This requires the use of oversized trocars and removal of the suture attachment through the trocar. The disposable sleeve must also be captured and withdrawn through the trocar.

Staple line reinforcement devices are commercially available from W. L. Gore & Associates, Inc., Flagstaff, Ariz., under the tradename SEAMGUARD®. Such staple line reinforcement devices are described in U.S. Pat. Nos. 5,702,409 and 5,810,855 to Rayburn et al. These devices comprise a material formed into a sleeve, which is sized to slip-fit over a stapler jaw or anvil. The sleeve incorporates tear lines or other means to allow easy separation of the disposable portions of the device, from the portions secured by the fired staples. Retrieval means, such as a suture, capture and allow retrieval of the disposable portions of the device.

In laparoscopic procedures, there are concerns similar to those discussed above with regard to U.S. patents to Cooper. After the staples have been fired and the conjoined tissue sections severed and reinforced by the SEAMGUARD® material, the excess material must be trimmed from the tissue sections and removed from the trocar before completing the surgical procedure.

In case of circular staples anastomoses (such as in bariatric surgery), the SEAMGUARD® was found to reduce post-operative stricture, yet it did not reduce bleeding or leaks [Early Clinical Results Using GORE SEAMGUARD® Bioabsorbable Staple Line Reinforcement For Circular Staplers; May 2007; Wesley B Jones MD, Katherine M Myers CST, Eric S Bour MD FACS].

An alternative staple line reinforcement device is commercially available from Synovis Inc., Saint Paul, Minn. under the tradename PERI-STRIPSDRY®. U.S. Pat. Nos. 5,752,965 and 5,810,855 to Francis et al. describe such a reinforcement device and a carrier used to present and load the device onto a stapler. This reinforcement material, comprising dried and treated bovine pericardium, is in the form of a strip sized to cover the desired part of the stapler. One or two of these pericardial strips are releasably attached to the carrier. Just prior to use, an adhesive gel is applied to the pericardial strips. The gel softens the strips and acts as an adhesive to allow temporary attachment to the stapler. The stapler is then self-aligned to the carrier, the jaws are closed upon the pericardial strips, and the gel adheres the strips to the stapler jaws. Unlike the slip-fit tubes of other reinforcement devices, the pericardial strips do not surround the stapler jaws. In order to provide for application of the strips, Francis et al. teach use of an apparatus having multiple deep guide channels to self-direct the surgical fastener into contact with the reinforcement material, and integral pressure equalization means in the form of resilient foam or similar material attached to the receiving area of the applicator card to aid in establishing a uniform adherence of the reinforcement strips to the surgical fastener.

There are a number of serious deficiencies with the Francis et al. apparatus. First, the use of bovine pericardium material is undesirable since this material requires preparation prior to use and must be kept moist to prevent embrittling and cracking when the staples are fired. Thus staples must be fired soon after mounting of the reinforcement material, limiting the ability to prepare multiple staplers with reinforcement devices prior to use. The implantation of bovine material also raises concerns associated with bovine maladies that can be transmitted to humans, such as Creutzfelt-Jakob Disease (CJD) or Bovine Spongiform Encephalopathy (BSE). Second, the carrier apparatus of Francis et al. may function adequately well for its intended purpose, but it is believed to be overly bulky in design due to the requirement for deep perpendicularly mounted guide channels.

Additionally, the apparatus of Francis et al. does not optimize material adherence to the surgical stapler. For instance, the method of attachment of the reinforcement material to the stapler arms is difficult to engineer among a variety of staple arm designs, thus requiring use of an integral layer of resilient foam to attempt to compensate for inaccurate sizing. Not only does the pressure equalizing foam provide less than optimal adherence, but due to the fact that Francis et al. teach that the foam is removed along with the reinforcement material upon application, additional steps are required for the surgical staff to remove and discard the foam prior to the insertion of the stapler into the patient.

Staple-line reinforcement strips from various biocompatible material are also described in U.S. Pat. No. 6,939,358 which discloses a self-adherent synthetic biocompatible material which is attached to an operational surface of a surgical stapler by an application card provided with pre-cut tear lines that allow the material to be applied held in place on the stapler while the surgical procedure is carried out, and then to buttress the surgical suture lines.

U.S. Pat. No. 6,656,193 discloses several buttress devices configured to engage surgical stapler jaw ends. These devices are configured for mechanical retention to the jaws until the stapling procedure has been completed.

U.S. Pat. No. 6,656,193 discloses a pericardial buttress strip provided with at least one end having an aperture for engaging at least on jaw end of the stapler.

U.S. Pat. No. 6,704,210 discloses a sealing film strip attached to a surgical stapler by passing a jaw of the stapler though openings formed in the ends of the strip.

In another example of a surgical system in which sealing of a vessel is important, management of bleeding at the femoral vascular access site following percutaneous catheterization is of paramount importance.

Traditionally, manual or mechanical compression has been the standard approach to achieve hemostasis. In many medical institutions, care protocol directed that a sandbag be used to compress a vascular access site for 4-6 hours following the removal of a catheter. Unfortunately, this method has many shortcomings. First, the process is time consuming, labor intensive and costly because it involves several hours of in-hospital observation. Second, sandbag compression is not desirable because the patient must remain immobilized for an extended period of time to avoid local hematoma formation. Third, extended compression can increase the risk of arterial occlusive complications. Hypertension and obesity can further complicate the procedure. Fourth, the required cessation of daily anticoagulation therapy prior to cardiac catheterization increases the risk of procedural complications (Nader et al. Journal of Invasive Cardiology 2002;14(6):305-307.).

Nonetheless, proper vascular closure is vital since vascular complications after femoral artery catheterization add significant morbidity to the procedure lengthen hospital stay and, in some cases, require blood transfusion and/or surgical repairs (Smith T P. Am J Surg 2001;182:658-662.).

In an effort to improve post-catheterization vascular closure, a number of vascular closure devices have entered the market in the past several years. These devices are intended to allow the removal of the sheath in a timely manner, decrease the time to hemostasis following diagnostic and interventional procedures, and decrease the patient time to ambulation. Examples of such devices include Perclose™ (Abbot Vascular Devices), Angio-Seal™ (St. Jude Medical), Therus™ (Boston Scientific), Duett™ (Vascular Solutions). Generally, such devices are used to close catheter holes with puncture sizes in the range of 5-8 French (F).

However, a number of reports on the use of vascular closure devices have documented serious complications, such as femoral artery or groin infections, related to use of certain closure devices (Johanning J M. J Vasc Surg 2001;34:983-985). In some cases, complications have led to limb amputation or death. Complication rates have a direct impact on patient satisfaction, the ability to maintain the femoral access site for future interventions, clinical outcomes and incremental costs associated with treating complications (Eidt, et al. Am J Surg 1999:178:511-516.). Unfortunately, the use of any of these existing invasive vascular closure devices precludes re-intervention at the same site for extended periods of time (Toursarkissian B, et al. Vasc Endovasc Surg 2001;35:203-206.).

In light of the complications associated with vascular closure devices, medical institutions tend to forego use of these devices in favor of assisted compression devices to assist in the closer of smaller puncture holes (<6 F). Assisted compression devices, in contrast with vascular closure devices, do not physically close the arterial wall puncture wound. However, they improve the efficacy of compression in closing catheter exit wounds either by mechanically maintaining compression, such as with EZ Hold™ (TZ Medical) or FemoStop™ (RADI Medical Systems), or by introducing a hemostatic material to the wound surface, such as with Chito-Seal™ (Abbot Vascular Devices), Neptune™ (TZ Medical), or D-Stat™ (Vascular Solutions). While assisted compression devices are not associated with surgical complications, their efficacy is also limited as they only moderately improve upon the technique of manual compression. For catheter exit wounds that would require 30 minutes of manual compression, assisted compression devices can reduce the treatment time to 15 minutes (Nader et al. Journal of Invasive Cardiology 2002;14(6):305-307). Furthermore, as noted above, the effective use of assisted compression devices is limited to smaller puncture holds.

Given the complications associated with vascular closure devices and the limited effectiveness of assisted compression devices, there remains a distinct need for a simple device for the control of bleeding at the femoral vascular access site following percutaneous catheterization. Such a device should be easy to use, effectively result in the stopping of bleeding from the access site, and not result in any surgical complications.

Sealants have been proposed as a solution to the problem of leakage from blood vessels but unfortunately all are currently defective. Fibrin sealant has been used clinically in the prevention of leak; however, its efficacy has not been clearly demonstrated.

Commercially, tissue adhesives of fibrin are derived from human plasma and thus raise potential risks to human health. Fibrin (and its derivatives) has been used in formulating biomedical adhesives with variable results from the experimental point of view and prospective studies in humans cannot be done. It is the only adhesive of use that is more or less accepted, but it is neither popular nor routine. Furthermore, fibrin has many disadvantages: risk of viral transmission; use of fibrin requires processes for extraction of blood; costs associated with fibrin are high; it requires a special applicator; risk of allergic reactions is always present; and a fatality has been reported. Another disadvantage of fibrin is that adhesion to tissue is relatively weak compared to other adhesives.

Other known sealants include synthetic PEG polymer, which show very weak adhesive strength, and BioGlue™ (albumin and gluteraldehyde), which is strong yet toxic.

Another problem of a surgical system which involves a bodily vessel is lymphorrhea. Prevention of lymphorrhea is generally required after surgical lymph-node dissection (LND) as part of the surgical treatment of different benign and malignant diseases such as: breast cancer, malignant melanoma, genito-urinary tumors, gastro-intestinal tumors, lung tumors, mediastinal tumors, ENT tumors. These surgical procedures may include: auxiliary LND, groin LND, neck LND, pelvic and retroperitoneal LND or any pelvic and retroperitoneal dissections, mediastinal LND, various vascular surgical interventions, various orthopedic interventions, etc. After such surgery, transected lymph vessels continue to drain lymph from the transected orifices, a process referred to as lymphorrhea. Nowadays, lymphostasis is achieved by tissue ligation or suturing, or alternatively requires a long period of constant observation until the lymphorrhea ceases. These procedures usually require mechanical drainage for several days, usually with hospitalization. Failure to drain the lymphorrea may result in lymph collection in the surgical wound, increases the risk of wound infection, may cause pain, swelling and severe inconvenience. On the other hand, fast lymphostasis will shorten hospitalization time and decrease risk of infection.

Cerebro-spinal fluid (CSF) leakage occurs in about 10% of cases after brain or spinal surgery, and frequently results in dangerous post-operative morbidity including meningitis with delayed neurologic complications, compression of neural structures, interference with wound healing, abscess formation, additional procedures, and prolonged hospitalization. (Source: Surgical Neurology 64 (2005) 490-494 “Healthcare Economics Costs of postoperative cerebrospinal fluid leakage: 1-year, retrospective analysis of 412 consecutive nontrauma cases” J. André Grotenhuis, MD, PhD). Pressure of the CSF can vary between 12-15 mmH.

DuraSeal®, a poly-ethylene glycol (PEG) polymer sealant, is the only dural sealant approved in the United States for cranial use. Use of DuraSeal in dura reconstruction surgeries has reduced the incidence of cerebro-spinal fluid leakage to about 4%, yet it has not succeeded in decreasing the infection rate. The lack of mechanical strength of the PEG sealant prevents it from being used more widely. In addition, DuraSeal is very expensive, having a unit price of $495/surgery.

Air leak is a major contributor to increased length of stay and postoperative morbidity following pulmonary surgery. The only FDA approved sealant for achieving pneumostasis is FocalSeal (Genzyme, Inc., Boston, USA). This sealant is a photopolymerized synthetic PEG hydrogel, which was taken off the market, probably due to its minor efficacy and the complications involved in using capital equipment (photo polymerizing lamps).

Surgeons have expressed a need for a fast, strong adhesive that is safe for use inside the body and resorbs as natural healing occurs. Such an adhesive could be used to adhere soft tissues in orthopedic applications, or to secure implants such as hernia meshes. Currently, the staples used to secure hernia mesh often lead to surgical trauma resulting in neuralgia and paresthesia due to nerve entrapment.

A tissue glue can be useful on planar surfaces, binding tissue layers (such as skin grafts) to eliminate the potential space between recently separated tissues in which fluid accumulates (potentially reducing the need for fluid drains).

Another use for a non-toxic tissue adhesive is for treatment of retinal detachment. The retina is a thin sheet of tissue (250-200 m) [Shahidi M, Blair N P, Mori M. Gieser J. Pulido J S. Retinal topography and thickness mapping in atrophic age related macular degeneration. Br J phthalmol 2002;86:623-626] consisting of nine separate tissue layers and several layers of cells. The outer segments of the retina's photoreceptors rest on a monolayer of retinal pigment epithelial (RPE) cells that separate the retina from the choroidal blood supply.

The RPE is integral to meeting the needs of the photoreceptors for nutrients and oxygen, and forms the blood-retinal barrier. There are no anatomical junctions anchoring the retina to the RPE. Rather, the retina is apposed to the RPE through a combination of metabolic and mechanical mechanisms that are not yet fully understood [Ghazi N G, Green W R. Pathology and pathogenesis of retinal detachment. Eve 2002;16:411-421. Marmor M F. Mechanisms of Normal Retinal Adhesion. In:Wilkinson C P, editor. Retina, Vol 3, 3rd edition. Philadelphia, Pa.: Mosby; 2001. p 1849-1869]

As a result of disease or injury, the retinal photoreceptors can be detached from the RPE, and because the RPE is vital to the physiology of the retina, reattachment is essential to preserve sight. [Steidl S M. Retinal Detachment. In: Steidl S M, Hartnett M E, editors. Clinical pathways in vitreoretinal disease, New York: Thieme; 2002.]

When a retinal break occurs in the absence of detachment, retinal attachment is preserved by creating a permanent retinal adhesion around the break. This is accomplished by exposing the RPE to a circular area of laser light, 50-500 microns in diameter, which induces a thermal reaction, resulting in tissue photocoagulation. This exposure to laser light is repeated in a pattern outlining the retinal defect, leading to a water-tight retinal seal.

For the first three days after laser treatment, weak adhesion is created from a proteinaceous coagulum that is replaced by a strong inflammatory-based scar beginning at about day 5 [Powell J O, Bresnick G H, Yanoff M, Frisch G D, Chester J E. Ocular effects of argon laser radiation. II. Histopathology of chorioretinal lesions. Am J Ophthalmol 1971;71:1267-1276.] Large or complex retinal detachments require more elaborate surgeries, such as vitrectomy or scleral buckle surgery.

During a vitrectomy, vitreous gel may be cut and aspirated, typically using a microsurgical, 20-gauge mechanical cutter with active suction. This is followed by fluid removal from the subretinal space to replace the retina against the RPE. An endolaser probe is then required to thermally induce tissue photocoagulation that develops into inflammatory-based scars that permanently attach the retina to the RPE.

To ensure the retina is forced in contact with the RPE, the surgeon often must fill the vitreous cavity with a tamponade material. Retinal tamponades are either long-acting gases (e.g., C3F8) or silicone oils. Unfortunately, the scar-forming lesions require 5 to 10 days to form, during which time the patient must maintain a head-down position.

Currently, there are two limitations of vitrectomy surgery for retinal reattachment.

First, the use of tamponades (gas or silicone oil) can lead to serious complications (e.g., cataracts and glaucoma). [Chang S. Intraocular Gases. In: Ryan S, editor. Retina. 3^{rd} edition. St. Louis, Mo.: Mosby; 2001; p 2147-2161; Abrams G W, Swanson D E, Sabates W I, Goldman A I. The results of sulfur hexafluoride gas in vitreons surgery. Am J Ophthalmol 1982;94:165-171; Barr C C, Lai M Y, Lean J S, Linton K L, Trese M, Abrams G, Ryan S J, Azen S P. Postoperative intraocular pressure abnormalities in the Silicone Study. Silicone Study Report 4. Ophthalmology 1993;100:1629-1635; Abrams G W, Area S P, Barr C C, Lai M Y, Hutton W L, Trese M T, Irvine A, Ryan S J. The incidence of conical abnormalities in the Silicone Study. Silicone Study Report 7. Arch Ophthalmol 1995;113:764-769; Karel I, Dotrelova D, Kalvodova B. Kalvodova J. Complicated cataract following intravitreal silicone oil injection and its surgery. In: Weidmann P, Heimann K, editors. Proliferative vitreoretinopathy.Heidelberg: Kaden-Verlag; 1988; Ando F. Intraocular hypertension resulting from pupillary block by silicone oil. Am J Ophthalmol 1985;99:87-88; Stefansson E. Anderson M M Jr, Landers M B III, Tiedeman J S, McCuen B W II. Refractive changes from use of silicone oil in vitreous surgery. Retina 1988;8:20].

Second, the postoperative requirement for head-down positioning for multiple weeks is uncomfortable for all patients, while compliance is impossible for some (e.g., victims of severe trauma and patients with neck and back problems).

An adhesive that confers short-term bonding of the retina and RPE while scars are forming would allow the surgeon to firmly reattach the retina during surgery. Such an adhesive could minimize the need and complications associated with intraocular tamponades, and eliminate the necessity for extended head-down positioning. While there has been considerable effort to develop ophthalmic adhesive, ultimately none have proved to be successful [Margalit E, Fujii G Y, Lai J C, Gupta P, Chen S J, Shyu J S, Piyathaisere D V, Weiland J D, De Juan E Jr, Humayun M S. Bioadhesives for intraocular use. Retina 2000;20:469-477; Schena L. Beyond Superglue: The Search for a Better Sealant. Eyenet 2003, January, 21-23; Bloom J N, Duffy M T, Davis J B, McNally-Heintzelman K M. A light-activated surgical adhesive for sutureless ophthalamic surgery, Arch Ophthamol 2003;121:1591-1595; Velazquez A J, Carnahan M A, Kristinsson J, Stinnett S. Grinstaff M W, sKim T. New dendritic adhesives for sutureless ophthalmic surgical procedures: in vitro studies of corneal laceration repair. Arch Ophthalmol 2004;122:867-870; Alio J L, Mulet M E, Cotlear D, Molina Y, Kremer I. Martin J M. Evaluation of a new bioadhesive copolymer (ADAL) to seal corneal incisions. Cornea 2004;23:180-189; Hoffman G T, Soller E C, Bloom J N, Duffy M T, Heintzelman D L, McNally-Heintzelman K M. A new technique of tissue repair for ophthalmic surgery. Biomed Sci Instrum 2004;40:57-63; Alio J L. Gomez J, Mulet E, Bujanda M M, Martinez J M, Molina Y. A new acrylic tissue adhesive for conjunctival surgery: experimental study. Ophthalmic Res 2003, 35:306-312].

There are several concerns with cyanoacrylate adhesive which are used in clinical practices: they react on contact with water, which complicates their delivery to moist intraocular surfaces, their ingredients and breakdown products can be toxic or not biocompatible, and their bonds can be too stiff and brittle for soft-tissue applications.

Two biologically-derived alternatives to cyanoacrylate adhesives have been considered for ophthalmic applications. The mussel glue is derived from the mussel's adhesive protein that is cured by an enzyme-initiated crosslinking reaction [Ninan L, Monahan J, Stroshine R L, Wilker J J, Shi R. Adhesive strength of marine mussel extracts on porcine skin. Biomaterials 2003;24:4091-4099; Strausberg R L, Link R P. Protein-based medical adhesives, Trends Biotechnol 1990;8:53-57; Olivieri M P, Baier R E, Loomis R E. Surface properties of mussel adhesive protein component films. Biomaterials 1992,13:1000-1008.]

In initial investigations for ophthalmic applications, the mussel glue elicited considerable inflammation and offered limited adhesive strength [Liggett P E, Cano M, Robin J B, Green R L, Lean J S. Intravitreal biocompatibility of mussel adhesive protein. A preliminary study. Retina 1990;10:144-147].

The second biological alternative is the fibrin sealants, which are currently used in some clinical settings and have been reported to be well tolerated in ophthalmic applications. [Kaufman H E, Insler M S, Ibrahim-Elzembely H A, Kaufman S C. Human fibrin sealant tissue adhesive for sutureless lamellar keratoplasty and scleral patch adhesion. Ophthamol 2003;110: 2168-2172.] However, the use of fibrin sealants in such applications appears to be limited, because their adhesive bonds are weak especially when curing (i.e., crosslinking) occurs under wet conditions.

Yet another use for a non-toxic tissue sealant is for tissue volume reduction, for example, lung volume reduction.

Patients with emphysema currently have limited treatment choices. Many patients are treated with steroids and inhaled medications, which often provide little or no benefit. In recent years, lung volume reduction surgery (LVRS) has become an accepted therapy for advanced emphysema. LVRS involves the removal of diseased portions of the lung in order to enable the remaining, healthier portions of the lung to function better (see, e.g., Cooper et al., J. Thorac. Cardiovasc. Surg. 109:106-116, 1995). While it may seem counter-intuitive that respiratory function would be improved by removing part of the lung, excising over-distended tissue (as seen in patients with heterogeneous emphysema) allows adjacent regions of the lung that are more normal to expand. In turn, this expansion allows for improved recoil and gas exchange. Even patients with homogeneous emphysema benefit from LVRS because resection of abnormal lung results in overall reduction in lung volumes, an increase in elastic recoil pressures, and a shift in the static compliance curve towards normal (Hoppin, Am. J. Resp. Crit. Care Med. 155:520-525, 1997).

While many patients who have undergone LVRS experience significant improvement (Cooper et al., J. Thorac. Cardiovasc. Surg. 112:1319-1329, 1996), substantial risk is involved. LVRS is carried out by surgically removing a portion of the diseased lung, which has been accessed either by inserting a thoracoscope through the chest wall or by a more radical incision along the sternum (Katloff et al., Chest 110:1399-1406, 1996). Thus, gaining access to the lung is traumatic, and the subsequent procedures, which can include stapling the fragile lung tissue, can cause serious post-operative complications.

Yet another use for a non-toxic sealant is in bridging gaps of lesioned nerves after peripheral or spinal injury. Current implants approved for human application do not allow regeneration across gaps of more than a few centimeters in length, possibly due to insufficient blood vessel formation (angiogenesis).

Yet another use for a sealant is to prevent post operative adhesions. Adhesions are caused by a scar that forms an abnormal connection between two parts of the body, causes by any trauma within the body as a consequence of normal healing (surgery, endometriosis, infection, radiation). Adhesions causes severe problems such as: infertility, chronic abdominal and pelvic pain, dyspareunia, bowel obstruction, complications in subsequent surgery, coalesce into Complex Abdomino-Pelvic and Pain Syndrome (CAPPS).

Adhesion-related disease (ARD) is underestimated and unappreciated. ARD admissions rival those for CABG, appendix, etc. In women undergoing gynaecological surgery, about 33% will be admitted about 2 times in the next 10 years for a problem directly related to adhesions or for procedure that could be complicated by adhesions (open or closed); pelvic adhesions found in 56-10% of patients undergoing second look laparoscopy; tubo-ovarian adhesions are a recognized cause of infertility and contribute to ectopic pregnancies. Adhesions related intestinal obstruction accounts for: 0.9% of all admissions; 3.3% of major laparotomies; 28.8% cases of L or S bowel intestinal obstruction.

### SUMMARY OF THE INVENTION

There is a need for, and it would be useful to have, an improved biocompatible medical sealant which is devoid of at least some of the limitations of the background art.

According to some embodiments of the present invention, there is provided a biocompatible medical sealant for use in a biological system, the sealant comprising a solution of a cross-linkable protein or polypeptide and a solution of a non-toxic cross-linking material which induces cross-linking of the cross-linkable protein, thereby sealing or adhering at least a portion of the biological tissue. The sealant preferably has suitable physiological properties to enable it to function well as a medical sealant. The non-toxic cross-linking material preferably comprises an enzymatic cross-liner. The cross-linkable protein or polypeptide is preferably not fibrin. Therefore the sealant is preferably an enzyme-crosslinked non-fibrin sealant.

The non-fibrin sealant optionally and more preferably has at least the following features, although this list is not intended to be limiting in any way; it is possible that the sealant has one or more additional features, or even lacks one or more features in the list: no protease inhibitor; single stage enzymatic reaction; can be cofactor independent; can be entirely non blood derived proteins.

According to some embodiments of the present invention there is provided use of a biocompatible medical adherent composition in a biological system, the composition comprising a non-fibrin cross-linkable polymer and an enzyme which induces cross-linking of the cross-linkable polymer, for thereby adhering at least a portion of the biological tissue, for reinforcement of surgical repair lines.

Optionally the surgical repair lines comprise one or more of staple lines and suture lines.

According to some embodiments of the present invention there is provided use of a biocompatible medical adherent composition in a biological system, the composition comprising a non-fibrin cross-linkable polymer and an enzyme which induces cross-linking of the cross-linkable polymer, for thereby adhering at least a portion of the biological tissue, for preventing anastomic dehiscence.

According to some embodiments of the present invention there is provided use of a biocompatible medical sealant in lung tissue, the sealant comprising a non-fibrin cross-linkable polymer and an enzyme which induces cross-linking of the cross-linkable polymer, for thereby sealing or adhering at least a portion of the lung tissue, for one or more of inducing pneumostasis or sealing lung tissue.

According to some embodiments of the present invention there is provided use of a biocompatible medical sealant in a dura tissue, the sealant comprising a non-fibrin cross-linkable polymer and an enzyme which induces cross-linking of the cross-linkable polymer, for thereby sealing or adhering at least a portion of the dura, for dura sealing.

According to some embodiments of the present invention there is provided use of a biocompatible medical sealant in biological tissue, the sealant comprising a non-fibrin cross-linkable polymer and an enzyme which induces cross-linking of the cross-linkable polymer, for thereby sealing or adhering at least a portion of the biological tissue, for one or more of sealing around an insertion wound into the biological tissue made by insertion of an implant or due to withdrawal of the implant.

Optionally the medical device comprises a catheter. Optionally the use is provided for the management of bleeding at a vascular access site following percutaneous catheterization.

Optionally the sealant is applied to the skin interface of the vascular access site.

Optionally manual pressure is applied to the surface of the vascular access site.

Optionally the pressure is applied for from about 5 to about 10 minutes.

Optionally the tissue is a blood vessel.

Optionally the sealing is performed following removal of the device from the tissue.

Optionally the medical device is a permanent device, and the sealing is performed around the device. Optionally the permanent device is a stoma tube.

Optionally the implant is selected from the group consisting of a soft tissue, tissue scaffold, a prosthesis and a skin graft. Optionally the prosthesis is a hernia mesh.

Optionally the tissue scaffold is used for curing myocardial infarction scars in heart tissue. Optionally the tissue scaffold is used for reconstruction of injured neural tissue in the peripheral or central nerve systems. Optionally the tissue scaffold polymerizes in-situ with cells. Optionally the cells are stem cells.

According to some embodiments of the present invention there is provided use of a biocompatible medical sealant in biological tissue, the sealant comprising a non-fibrin cross-linkable polymer and an enzyme which induces cross-linking of the cross-linkable polymer, thereby sealing or adhering at least a portion of the biological tissue, for attaching the biological tissue to an artificial material.

According to some embodiments of the present invention there is provided use of a biocompatible medical sealant in biological tissue, the sealant comprising a non-fibrin cross-linkable polymer and an enzyme which induces cross-linking of the cross-linkable polymer, for forming a cell scaffold in situ through polymerization of the protein material due to cross-linking.

According to some embodiments of the present invention there is provided use of a biocompatible medical sealant in biological tissue, the sealant comprising a non-fibrin cross-linkable polymer and an enzyme which induces cross-linking of the cross-linkable polymer, thereby sealing or adhering at least a portion of the biological tissue, for closing a fistula.

According to some embodiments of the present invention there is provided use of a biocompatible medical sealant in biological tissue, the sealant comprising a non-fibrin cross-linkable polymer and an enzyme which induces cross-linking of the cross-linkable polymer, thereby sealing at least a portion of the biological tissue, for preventing adhesion of the biological tissue to another biological tissue.

According to some embodiments of the present invention there is provided use of a biocompatible medical sealant in lung tissue, the sealant comprising a non-fibrin cross-linkable polymer and an enzyme which induces cross-linking of the cross-linkable polymer, thereby sealing or adhering at least a portion of the lung tissue, for Biological Lung Volume Reduction.

Optionally the cross-linkable polymer comprises a non-fibrin protein. Optionally the non-fibrin protein comprises gelatin. Optionally the enzyme is selected from the group consisting of calcium dependent or independent transglutaminase, tyrosinase and laccase. Optionally the enzyme comprises microbial transglutaminase.

Optionally the composition further comprises a transition point lowering agent for lowering the gelatin transition point.

According to some embodiments of the present invention there is provided use of a biocompatible medical sealant in biological tissue, the sealant comprising gelatin, a transition point lowering agent for lowering the gelatin transition point, and microbial transglutaminase which induces cross-linking of the gelatin, thereby sealing or adhering at least a portion of the biological tissue, for inducing one or both of hemostasis or lymphostasis.

Optionally the lymphorrhea occurs after surgical lymph-node dissection.

Optionally the surgical lymph node dissection is selected from the group consisting of auxiliary surgical lymph-node dissection, groin surgical lymph-node dissection, neck surgical lymph-node dissection, and pelvic and retroperitoneal surgical lymph-node dissection.

According to some embodiments of the present invention there is provided use of the biocompatible medical sealant in a high pressure biological system for an application selected from the group consisting of fortification of vascular anastomosis and grafts, hemostasis of injured arteries, veins, and fluid-stasis in parenchimatic organs.

According to some embodiments of the present invention there is provided use of a biocompatible medical sealant in lung tissue, the sealant comprising gelatin, a transition point lowering agent for lowering the gelatin transition point, and microbial transglutaminase which induces cross-linking of the gelatin, thereby sealing or adhering at least a portion of the lung tissue, for Biological Lung Volume Reduction.

According to some embodiments of the present invention there is provided use of a biocompatible medical sealant in biological tissue, the sealant comprising gelatin, a transition point lowering agent for lowering the gelatin transition point, and microbial transglutaminase which induces cross-linking of the gelatin, thereby sealing or adhering at least a portion of the biological tissue, for sustained therapeutic agent release from the sealant.

According to some embodiments of the present invention there is provided use of a biocompatible medical sealant in ocular tissue, the sealant comprising gelatin, a transition point lowering agent for lowering the gelatin transition point, and microbial transglutaminase which induces cross-linking of the gelatin, thereby sealing or adhering at least a portion of the ocular tissue, for retinal attachment.

According to some embodiments of the present invention there is provided use of a biocompatible medical sealant in a biological tissue, the sealant comprising gelatin, a transition point lowering agent for lowering the gelatin transition point, and microbial transglutaminase which induces cross-linking of the gelatin, thereby sealing or adhering at least a portion of the neurological tissue or sealing a cerebrospinal fluid leak.

Optionally the cerebro-spinal fluid leakage occurs due to a surgical procedure selected from the group consisting of brain surgery or injury and spinal surgery or injury.

According to some embodiments of the present invention there is provided use of the biocompatible medical sealant wherein the sealant further comprises at least one additional protein or polypeptide.

According to some embodiments of the present invention there is provided use of the biocompatible medical sealant for sustained release of a biologically active peptides and proteins incorporated in the sealant.

According to some embodiments of the present invention there is provided use of the sealant for delivering a therapeutic agent.

Optionally the therapeutic agent comprises an antibiotic and/or an anesthetic.

According to some embodiments of the present invention there is provided use of the biocompatible medical sealant, wherein the sealant further comprises at least one transition point-lowering agent selected from the group consisting of urea and calcium.

Optionally the sealant further comprises at least one selected from the group consisting of a calcium sequestering agent, a urea sequestering agent, a urea hydrolyzing agent and ammonia scavenging agent.

Optionally the sealant is applied in the form of a liquid, gel, spray, foam, or lyophilized form. Optionally the sealant further comprises a supportive bio-absorbable backing.

Optionally the sealant is dried together with the supportive bio-absorbable backing.

According to some embodiments of the present invention there is provided a non-surgical method of reducing lung volume in a patient, the method comprising: (a) collapsing a target region of the patient's lung; and (b) administering, by way of the patient's trachea, to the target region of the patient's lung: (i) a first composition comprising a gelatin and (ii) a second composition comprising a gelatin crosslinker, whereafter one portion of the target region adheres to another portion of the target region, thereby reducing the patient's lung volume.

Optionally the target region is collapsed by blocking air flow into or out of the region.

Optionally the target region is collapsed by lavaging the target region with an anti-surfactant.

Optionally the method is performed using a bronchoscope. Optionally the patient is a human patient. Optionally the patient has emphysema. Optionally the patient has suffered a traumatic injury to the lung.

According to some embodiments of the present invention there is provided a method of preparing the sealant, wherein the transition point reducing agent is removed or neutralized before application.

Optionally the transition point reducing agent comprises the application of heat to the gelatin.

According to some embodiments of the present invention there is provided use as described herein wherein gelatin and/or transglutaminase are stored in a lyophilized form and are mixed before use.

According to some embodiments of the present invention there is provided a system for applying a composition or sealant according to any of the above claims, comprising: a plurality of syringes connected to a central applicator, at least one syringe containing a non-fibrin cross-linkable polymer and at least one other syringe containing an enzyme which induces cross-linking of the cross-linkable polymer, wherein pressure upon the syringes causes their contents to enter the central applicator and to be mixed therein, for being applied to a biological tissue from the central applicator.

Optionally at least the syringe containing the polymer is heated before the pressure is applied, such that the polymer is heated before mixing with the enzyme.

The biocompatible medical sealant is optionally used in a biological system selected from the group consisting of a low pressure biological system and a high pressure biological system.

According to some embodiments, the sealant is used for reinforcement of surgical repair lines, such as staple lines (including those produced by an endoscopic stapler) and suture lines in a low pressure biological system.

According to some embodiments, the sealant of the present invention is used for providing fluid-stasis, including gas-stasis, hemostasis, and pneumostasis

Optionally fluid-stasis is provided for a surgical procedure selected from the group consisting of vascular reconstructions, dura reconstructions, thoracic, cardiovascular, lung, neurological, and gastrointestinal surgeries.

Also optionally, the fluid-stasis comprises lymphostasis.

According to some embodiments, the sealant is used for preventing lymphorrhea, such as that which occurs after surgical lymph-node dissection, including, but not limited to auxiliary surgical lymph-node dissection, groin surgical lymph-node dissection, neck surgical lymph-node dissection, and pelvic and retroperitoneal surgical lymph-node dissection.

According to some embodiments, the sealant is used for preventing cerebro-spinal fluid leakage, such as that which occurs due to a surgical procedure, such as brain surgery or injury and spinal surgery or injury.

According to some embodiments, the sealant is used in a high pressure biological system for an application such as fortification of vascular anastomosis and grafts, hemostasis of injured arteries, veins, and fluid-stasis in parenchimatic organs.

According to some embodiments, the sealant is used for sealing a puncture site for insertion of a medical device (such as a catheter) into a tissue (such as a blood vessel), for example, following removal of the device from the tissue, or around a permanent device (such as a stoma tube).

According to some embodiments, the sealant is used for sealing an attachment between a tissue and a material, wherein the material is, for example, a soft tissue, tissue scaffold, an implant, a prosthesis (such as a hernia mesh) and a skin graft.

According to some embodiments, the tissue scaffold is used, for example, for curing myocardial infarction scars in heart tissue, or for reconstruction of injured neural tissue in the peripheral or central nerve systems

According to some embodiments, the tissue scaffold polymerizes in-situ with cells, such as, for example, stem cells.

According to some embodiments, the sealant itself optionally forms a tissue scaffold through in situ polymerization, by cross-linking of the non-fibrin protein material or mixture thereof with an enzyme. Preferably the non-fibrin protein material comprises gelatin and the enzyme comprises transglutaminase.

According to some embodiments, the sealant is used for prevention of anastomotic dehiscence.

According to some embodiments, the sealant is used for sealing a fistula.

According to some embodiments of the sealant of the present invention, the non-toxic cross-linking material comprises an enzyme, such as, for example, transglutaminase (TG), tyrosinase or laccase, or mixtures thereof.

According to some embodiments of the sealant of the present invention, the cross-linkable protein comprises gelatin or collagen, or mixtures thereof.

According to any of the embodiments of the present invention, the gelatin may comprise recombinant gelatin.

According to some embodiments of the present invention, the sealant further comprises at least one transition point-lowering agent, such as, for example and without wishing to be limited, urea or calcium, or mixtures thereof.

According to some embodiments of the present invention, the sealant further comprises at least one of a calcium sequestering agent, a urea sequestering agent, a urea hydrolyzing agent and ammonia scavenging agent.

According to some embodiments of the present invention, there is provided a non-surgical method of reducing lung volume in a patient, the method comprising collapsing a target region of the patient's lung; and administering, by way of the patient's trachea, to the target region of the patient's lung a first composition comprising a gelatin and a second composition comprising a gelatin crosslinker, which is preferably transglutaminase but which in any case is enzymatic, wherein the composition does not feature fibrin, whereafter one portion of the target region adheres to another portion of the target region, thereby reducing the patient's lung volume. Optionally, a single composition may be administered rather than two separate compositions.

Optionally, the first composition comprises about 10-25% gelatin 175-300 bloom.

Also optionally, the first composition includes a gelatin transition point reducing agent.

According to some embodiments of this aspect of the present invention, the gelatin crosslinker is an oxidative enzyme or transglutaminase or a combination thereof.

According to some embodiments, the first or second composition further comprises an antibiotic.

According to some embodiments of this aspect of the invention, the target region is collapsed by blocking air flow into or out of the region.

According to some embodiments of this aspect of the invention, the target region is collapsed by lavaging the target region with an anti-surfactant.

Optionally, the method is performed using a bronchoscope.

Optionally, the patient is a human patient.

According to some embodiments of this aspect of the invention, the patient has emphysema.

According to some embodiments of this aspect of the invention, the patient has suffered a traumatic injury to the lung.

According to some embodiments. the sealant of the present invention further comprises at least one additional protein or polypeptide.

According to some embodiments. the sealant of the present invention is used for providing sustained release of a biologically active peptides and proteins incorporated in the sealant.

According to some embodiments of the present invention, there is provided a biocompatible medical adhesive for use in a biological system, the adhesive comprising a solution of a cross-linkable protein or polypeptide and a solution of a non-toxic cross-linking material which induces in-situ cross-linking of said cross-linkable protein, thereby creating a layer over the tissue.

Optionally, the layer is a protective layer. Further optionally, the layer is used for preventing post surgical tissue adhesions

According to some embodiments, the medical sealant of the present invention is used for repair of retinal detachment.

Unless defined otherwise, all technical and scientific terms used herein have the same meaning as is commonly understood by one of skill in the art to which this invention belongs. All patents, patent applications, and publications mentioned herein are incorporated herein by reference.

As used herein, the term “sealant” refers to a material which provides an intimate contact and elimination of space between a tissue and a material, including between two tissues. Sealing therefore includes closure of a tear, wound or puncture in a tissue, and attachment of a material such as a tissue, graft, implant or prosthesis to a tissue. Preferably, the sealant makes not only direct contact with the surface of the receiving tissue, but also penetrates into the hollows or grooves of the tissue so that mechanical, chemical and/or electrostatic connections or unions or links are formed. Optionally the tissue and the material contact each other only through the sealant, although this is not necessary.

As used herein, “about” means plus or minus approximately ten percent of the indicated value.

Other features and advantages of the invention will be apparent from the following detailed description, and from the claims.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic view of the eye; and FIG. 2b is a schematic illustration of vitrectomy surgery to reattach complex retinal detachments,

FIG. 2 is a schematic representation of the lungs;

FIG. 3a is a schematic representation of bleeding upon removal of a catheter from a blood vessel;

FIG. 3b is a schematic representation of application of a sealant to a vascular site upon removal of a catheter from a blood vessel;

FIG. 3c is a schematic representation of application of an adhesive to a vascular site upon removal of a catheter from a blood vessel;

FIG. 4 shows bronchitis obliterans of the lobe of the right lung induced by LifeSeal GS medical sealant in a pig after lung volume reduction; the black arrow indicates LifeSeal GS remnants, obstructing the main bronchus and bronchi of the lung;

FIG. 5 shows that the sealant according to the present invention prevents adhesion formation in rats; (A) Formation of adhesions in the control group (B) LifeSeal GS (indicated by a black arrow) covers the abrasion and prevents adhesion formation;

FIG. 6 shows that LifeSeal medical sealant adheres strongly to the dura tissue. A) The dura is revealed; B) LifeSeal sealant is applied onto the dura; C) LifeSeal sealant strongly adheres to the dura tissue and forms a stable biofilm that connects the two dura parts;

FIG. 7 shows photographs of the anastomosis model used for testing the sealant of the present invention. A) A staple is removed from the circular stapler; B) The staple line in the upper rectum; C) Air bubbles are formed in the abdomen cavity that is filled with water, after application of air pressure, demonstrating a leak;

FIG. 8 shows a comparison of the burst pressure results of LifeSeal SLR sealant compared to the control baseline leakage;

FIG. 9 shows buttressing of anastomosis staple line using LifeSeal SLR (HE staining; Magnitude—Macro). LifeSeal (black arrow) sealant adheres to the rectum's serosa tissue, securing the anastomosis staple line (holes of removed staples are indicated by yellow arrows). A newly formed granulation tissue (green arrow) that is composed mainly of fibroblasts is bridging the tissue parts. As indicated by this figure, LifeSeal does not interfere with the natural healing process. Pink arrows point out the mucosa tissue;

FIG. 10 is a line graph showing swelling of gels at 37° C. over a period of 5 hours;

FIG. 11 is a line graph showing swelling of gels at 37° C. over a period of 48 hours; and

FIG. 12 shows the results for the Bromophenol Blue concentrations released from the gels as function of time.

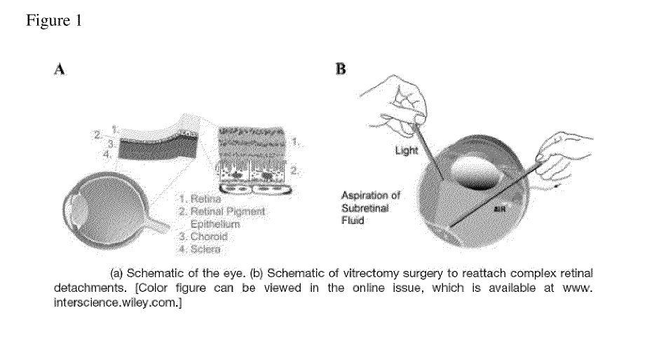

### DESCRIPTION OF THE PREFERRED EMBODIMENTS

The present invention provides a biocompatible medical sealant for use in a biological system, the sealant comprising enzyme-crosslinked non-fibrin sealant, which according to some embodiments comprises gelatin and transglutaminase, optionally with one or more additional components as described herein. The sealant may optionally be formed through the mixture of two or more compositions, such as two or more solutions, at the time of or shortly before or after administration. By “shortly before administration” it is meant preferably up to about 5 minutes, more preferably up to about 2 minutes and most preferably up to about 1 minute (for example, most preferably up to about 30 seconds).

By “administration” it is meant any type of contacting between the sealant and a tissue with one or more other materials, such as another tissue for example and/or one or more non-tissue materials, optionally including one or more artificial materials. Alternatively, the sealant may optionally be provided as a single composition, such as a single solution for example.

The biocompatible medical sealant of the present invention is not toxic, does not produce serious adverse reactions, and minimizes demands on surgical resources and time, coupled with a superior biocompatibility and biostability. It is safe, strong, biodegradable, and relatively cheap to manufacture. The precise composition of the sealant of the present invention can be adjusted such that the sealant sets at any desirable time.

The biological system may be a low pressure system or a high pressure system.

For low pressure systems, the sealant of the present invention is exceptionally useful for applications such as reinforcing surgical sutures and surgical staples, and/or for any type of sealant and/or adherent activity.

Post-operative leakage from staple or suture lines is a common complication of the conventional closure methods, associated with life-threatening morbidity.

As discussed in the Background section above, reinforcing strips for reduction of staple-line leaks are known. However, such strips are limited to use with staples and are not suitable for sutures.

Furthermore, background art strips such as the Peristrip (Synovis) and SURGISIS (Cook), which are made from a bovine source, have many limitations and shortcomings due to their source of material. Other known strips, such as the SEAMGUARD (Gore) and PeriPatch Aegis (PM Devices Inc.), are problematic due to their low efficacy. Application of all such strips are time consuming during surgery, because the strip must be installed on the staple each time the stapler is activated, and excess strip material must be removed. The strips may not optimally fit the stapled tissue line, leading to further difficulties and reduced utility.

According to some embodiments, the present invention provides a biocompatible medical sealant for use in reinforcement of surgical repair lines. The surgical repair lines may comprise, for example, staple lines or suture lines.

The biocompatible medical sealant of the present invention enables securing suture and staple line against leaks, regardless of the method used for the tissue approximation, and regardless of the morphology of the stapled tissue (i.e. whether linear or circular or any other form). The sealant is easy to prepare and to use, and requires the surgeon merely to apply the sealant to the general area to be sealed. The ease of use is especially relevant for minimal invasive surgery.

The biocompatible medical sealant of the present invention may be used on a stapler to fully protect surgical staple lines. The method may be used, for example, with an endoscopic stapler.

According to some embodiments, the biocompatible medical sealant of the present invention is used for preventing anastomotic dehiscence.

The term “dehiscence” used presently includes any defect or failure of the anastomosis in the gastrointestinal, respiratory, urinary systems, etc., that can produce leakage of secretions and bacteria through this defect, with very serious and frequently lethal consequences. This statement is not to be considered as limiting but rather illustrative of some of the applications of some embodiments of the biological adhesive of the present invention to eliminate dehiscences of anastomosis to the maximum.

Preferably, such use allows effective temporary protection of the anastomosis up to the eighth postoperative day. It is during this time period that the anastomosis is particularly weak because collagen deposition and development of new tissue bridges has yet to occur. Use of the sealant of the present invention offers improved resistance to leakage where applied, without affecting the original physiological functions of the digestive system and other organ systems.

The efficacy of the sealant of the present invention was demonstrated in Example 4, whereas it was demonstrated that the the sealant can adhere well to a living intestine of a pig.

According to some embodiments, the biocompatible medical sealant of the present invention is used for providing fluid-stasis, either alone, or as an adjunct to sutures or staples.

For example, the biocompatible medical sealant of the present invention is used to achieve hemostasis or other fluid-stasis in surgical procedures including, but not limited to, peripheral vascular reconstructions, dura reconstructions, thoracic, cardiovascular, lung, neurological, and gastrointestinal surgeries.

According to some embodiments, the biocompatible medical sealant of the present invention is used to provide lymphostasis.

According to some embodiments, the biocompatible medical sealant of the present invention is used for preventing lymphorrhea, such as, for example, that occurring after a surgical lymph-node dissection (LND), such as, for example, auxiliary LND, groin LND, neck LND, pelvic and retroperitoneal LND or any pelvic and retroperitoneal dissections, mediastinal LND, or various vascular surgical or orthopedic interventions.

According to one non-limiting example, the biocompatible medical sealant may be sprayed onto the region of a lymph node or transected lymph duct.

For use as a dura sealant, a material must not expand by more than about 100% after application, but preferably expands significantly less. DuraSeal®, a poly-ethylene glycol (PEG) polymer sealant is the only dural sealant currently approved in the United States for cranial use. Use of DuraSeal® in dura reconstruction surgeries has reduced the incidence of cerebro-spinal fluid leakage from about 10% to about 4%, yet has not succeeded in decreasing the infection rate.

The biocompatible medical sealant according to some embodiments of the present invention, featuring an enzyme-crosslinked non-fibrin sealant, has strong adhesive strength; water absorption and thus expansion in-situ is negligible (5-10%) as described in Example 3. Preferably, the sealant according to embodiments for use for dura sealing only expands up to about 30%, more preferably up to about 20% and most preferably up to about 10%.

According to some embodiments, the biocompatible medical sealant of the present invention is used for prevention of cerebro-spinal fluid (CSF) leakage.

The CSF leakage which is prevented by use of the biocompatible medical sealant according to some embodiments of the present invention may occur, for example, due to a surgical procedure such as brain or spinal surgery.

Air leak is a major contributor to increased length of stay and postoperative morbidity following pulmonary surgery and there is no current sealant in the market for this specific use. As said in the background the FocalSeal was FDA approved but was taken off market for various reasons. The enzyme-crosslinked non-fibrin sealant according to some embodiments of the present invention has strong adhesive strength to tissue and is capable of withholding pressure higher than that of the air inside the lung. It is also flexible enough to withstand the elastic forces of lung tissue during the breathing cycle. According the some embodiments, it is also possible to adjust the elasticity of the sealant according to the medical need by altering the composition.

Another embodiment of the invention features devices, compositions, and methods for achieving non-surgical lung volume reduction. In one aspect, the methods are carried out using a bronchoscope, which completely eliminates the need for surgery because it allows the tissue reduction procedure to be performed through the patient's trachea and smaller airways. In this approach, bronchoscopic lung volume reduction (BLVR) is performed by collapsing a region of the lung, adhering one portion of the collapsed region to another, and promoting fibrosis in or around the adherent tissue.

There are numerous ways to induce lung collapse. For example, a material that increases the surface tension of fluids lining the alveoli (i.e., a material that can act as an anti-surfactant) can be introduced through the bronchoscope (preferably, through a catheter lying within the bronchoscope). The material can include gelatin, or biologically active fragments thereof.

Similarly, there are numerous ways to promote adhesion between one portion of the collapsed lung and another. If gelatin is selected as the anti-surfactant, adhesion is promoted by exposing the gelatin to a non-toxic crosslinking activator, such as but not limiting to an oxidative enzyme or transglutaminase, which polymerizes the resulting gelatin. In this case, no additional substance or compound need be administered, apart from the crosslinking activator; gelatin can polymerize spontaneously upon induction of crosslinking, thereby adhering one portion of the collapsed tissue to another. Preferably, the crosslinking activator comprises transglutaminase.

Fibrosis is promoted by providing one or more polypeptide growth factors together with one or more of the anti-surfactant or activator substances described above. The growth factors can be selected from the fibroblast growth factor (FGF) family or can be transforming growth factor beta-like (TGF.beta.-like) polypeptides.

The compositions described above can also contain one or more antibiotics to help prevent infection. Alternatively or in addition, antibiotics can be administered via other routes (e.g., they may be administered orally or intramuscularly).

Other aspects of the invention include the compositions described above for promoting collapse and/or adhesion, as well as devices for introducing the composition into the body. For example, in one aspect, the invention features physiologically acceptable compositions that include a polypeptide growth factor or a biologically active fragment thereof (e.g., a platelet-derived growth factor, a fibroblast growth factor (FGF), or a transforming growth factor-.beta.-like polypeptide) and gelatin, or with gelatin transition agent (e.g., urea and calcium), or with a non-toxic crosslinking agent (e.g., transglutaminase). The gelatin, gelatin peptides, and gelatin crosslinkers useful in BLVR can be biologically active mutants (e.g., fragments) of these polypeptides.

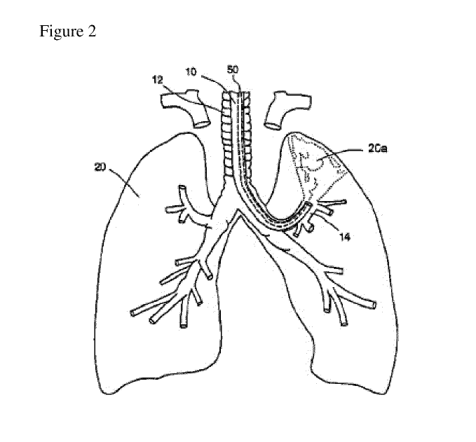

According to other embodiments, the invention features devices for performing non-surgical lung volume reduction. For example, the invention features a device that includes a bronchoscope having a working channel and a catheter that can be inserted into the working channel. The catheter can contain multiple lumens and can include an inflatable balloon. Another device for performing lung volume reduction includes a catheter having a plurality of lumens (e.g., two or more) and a container for material having a plurality of chambers (e.g., two or more), the chambers of the container being connectable to the lumens of the catheter. These devices can also include an injector to facilitate movement of material from the container to the catheter. The catheter can be heated (e.g., to 50 degree C.) by to facilitate more efficient movement of the gelatin into the lung (see FIG. 2).

BLVR has several advantages over standard surgical lung volume reduction (LVRS). BLVR should reduce the morbidity and mortality known to be associated with LVRS (Swanson et al., J. Am. Coll. Surg. 185:25-32, 1997). Atrial arrhythmias and prolonged air leaks, which are the most commonly reported complications of LVRS, are less likely to occur with BLVR because BLVR does not require stapling of fragile lung tissue or surgical manipulations that irritate the pericardium. BLVR may also be considerably less expensive than SLVR. The savings would be tremendous given that emphysema afflicts between two and six million patients in America alone. In addition, some patients who would not be candidates for LVRS (due, e.g., to their advanced age) may undergo BLVR. Moreover, should the need arise, BLVR affords patients an opportunity to undergo more than one volume reduction procedure. While repeat surgical intervention is not a viable option for most patients (because of pleural adhesions that form following the original procedure), no such limitation should exist for patients who have undergone BLVR.

U.S. Pat. No. 6,610,043 describes a method for undergoing BLVR using fibrin, fibrinogen and fibrinogen activator as the bioadhesive used for the anti-surfactant. This patent is currently being developed into product by Aeris Therapuetics, Inc., Woburn, Mass. and Omrix Inc., Ness Ziona, Israel is the supplier of the human derived fibrin. The many disadvantages of using fibrin include but are not limited to the unavoidable risk of viral transmission, the limited supply, the quality variability and the higher costs of manufacturing.

For high pressure systems, the biocompatible sealant of the present invention may be used, for example and without limitation, to fortify vascular anastomosis and grafts, or for hemostasis of injured arteries or veins, and for stasis of fluid oozing from injured parenchimatic organs.

The biocompatible medical sealant of the present invention is also useful in sealing a puncture site for introduction of a catheter or other medical device into the body. The sealant may be applied with an appropriate dispenser to the puncture site.

In an embodiment of the current invention, the biocompatible medical sealant composition of the present invention is used for the management of bleeding at a vascular access site following percutaneous catheterization.

According to some embodiments, the composition is applied to the skin interface of the vascular access site. The composition can be applied to the skin surface in liquid, gel, spray, foam, or lyophilized form. After the composition is applied, manual pressure is applied to the surface to facilitate strong adhesion. Preferentially, pressure is applied for 5-10 minutes until a strong bond is formed between the composition and the skin surface. The composition can then act to maintain pressure on the access site even once manual pressure has been removed.

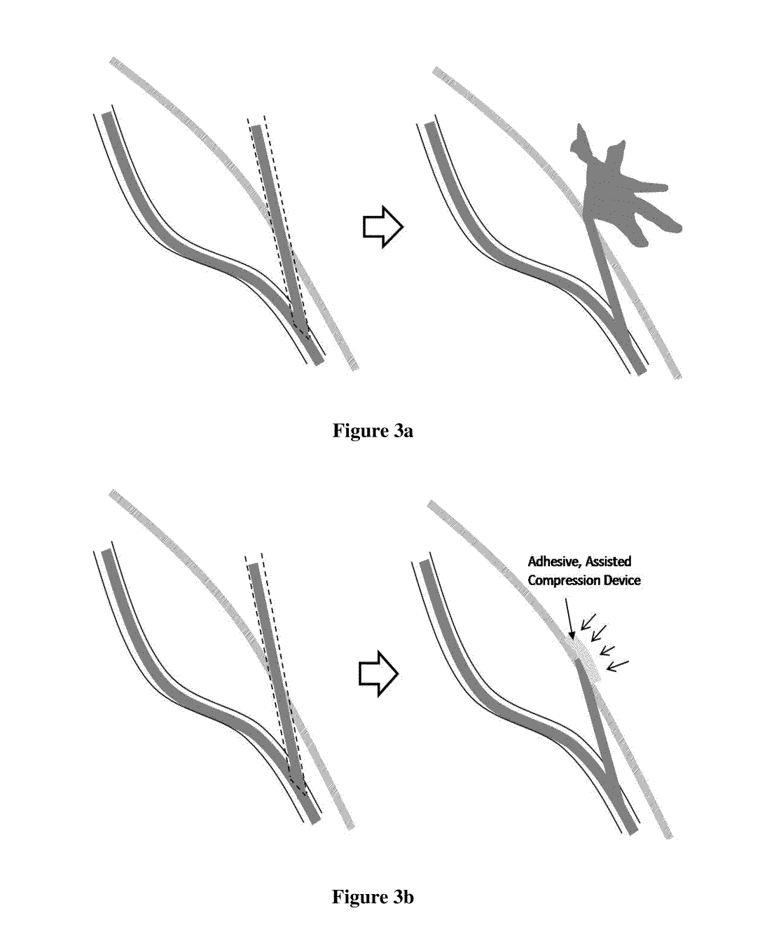

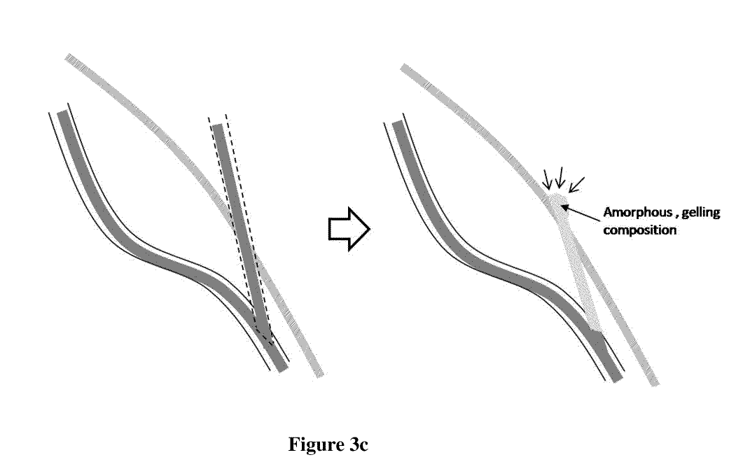

As shown in FIG. 3a, when the catheter is removed, bleeding occurs from the vascular access site.

As shown in FIG. 3b, the sealant of the present invention may be applied to the vascular access site when the catheter is removed, thereby closing the access site at the skin interface.

The application of the herein described biocompatible sealant composition for closure of a vascular access site at the skin interface is done in a procedure similar to the procedure that is used currently for the application of assisted compression devices, particularly assisted compression pads or patches. Examples of such types of pads or patches are Chito-Seal™ (Abbot Vascular Devices), V+ Pad™ (InterV), Syvek Patch™ (Marine Polymer Technologies), Clo-Sur Plus P.A.D.™ (Medtronic), StasysPatch™ (St. Jude Medical), Neptune™ (TZ Medical), and D-Stat™ (Vascular Solutions). However, the efficacy of existing pads and patches is limited as they are only nominally adhesive. The herein described application of a biocompatible sealant composition for skin surface closure of a vascular access site amounts to an adhesive, assisted compression device, which has vastly improved efficacy as the composition remains strongly stuck to the access site even once manual compression is removed.

According to a preferred embodiment of the current invention, the herein described biocompatible medical sealant composition is applied to the skin surface of the vascular access site. The composition can be applied for example optionally as any of liquid, gel, spray, foam, or lyophilized form. Once the composition is applied, strong pressure is applied to the composition to direct the composition down into the channel from which the catheter has been removed. As shown in FIG. 3c, the composition can then fill the catheter access channel, become anchored in the channel, and block exit of blood from the blood vessel into the catheter channel.

Preferably, the composition undergoes a process of gelation stemming from in situ cross-linking after entering the catheter channel to securely close the channel to further blood flow.

In some embodiments, a device is used to direct pressure onto the sealant composition to facilitate improved transfer of the composition into the catheter access site channel. Examples of pressure transduction devices for use with this application include bandages, tourniquets, tape, or any other device that can apply pressure to a vascular access site either by encircling the limb containing the access site, by adhering to skin surfaces around the access site, or by any other method of applying pressure to a wound site. A balloon or other method of increasing pressure can be incorporated in the pressure transduction device. An example of a pressure transduction device that incorporates a balloon that would be useful for the current application is Safeguard™ (Datascope).

These methods of vascular access site closure are useful for catheter puncture sizes from 1-10 F. Preferably, these methods are useful for catheter puncture sizes from 1-8 F.

Optionally, the medical sealant is applied to the puncture site once the device has been removed. For example, the sealant may be used for provide vascular closure following puncture of a blood vessel. To prevent the entrance of sealant into the blood vessel, the catheter can utilize a balloon or other mechanical method that will temporarily hold the puncture closed and allow the sealant components to react and set. Once set, the sealant will not enter the vessels and is capable of withstanding arterial pressure.

Alternatively, the sealant may be applied following insertion of a permanent device, such as a stoma tube. The sealant can secure the entry port and prevent the leakage of body fluids from around the tube. It will also prevent the entrance of infectious microorganisms into and around the port.

According to some embodiments, the biocompatible medical sealant may be used to seal and/or attach a tissue and a material, including but not limited another tissue, tissue scaffolds or other synthetic substances, including without limitation medical devices such as catheters or implants. The material may comprise, for example and without wishing to be limited, a soft tissue, an implant, a prosthesis, or a skin graft.

Recently a new product ARTISS (Baxter) was approved by the FDA, indicated for adhering autologous skin grafts to surgically prepared wound beds resulting from burns in adults and pediatric populations. ARTISS allows for the delayed setting and controlled manipulation of skin grafts for approximately 60 seconds, relative to rapid-setting fibrin sealants, which set in five to 10 seconds. Skin grafts can be fixed without the use of staples or sutures, which may help reduce post-operative complications and patient anxiety about pain during staple removal. Because ARTISS is made from human plasma, it may carry a risk of transmitting infectious agents, e.g., viruses, and theoretically, the Creutzfeldt-Jakob disease (CJD) agent. ARTISS cannot be used in individuals with a known hypersensitivity to aprotinin. Adverse reactions occurring in greater than 1% of patients treated with ARTISS were skin graft failure and pruritus.

By contrast, the medical sealant of the present invention is made from highly biocompatible materials, preferably an enzyme-crosslinked non-fibrin sealant, more preferably comprising gelatin, for example porcine, bovine, fish or recombinant human gelatin, more preferably cross-linked with an enzymatic cross-linker such as transglutaminase for example.

The biocompatible aspects of the medical sealant of the present invention make it useful for sealing tissue to tissue in a wide variety of different applications.

The sealant of the present invention can be useful, for example, on planar surfaces, sealing attachment of tissue layers (such as skin grafts) to eliminate the potential space between recently separated tissues in which fluid accumulates (potentially reducing the need for fluid drains).

The sealant of the present invention can be used for temporary fixation of prosthesis in hernia operations (such as inguinal hernias). In addition, the sealant can be used to facilitate the closing of some digestive fistulas or fistulas of other organ systems, if there is no obstruction or active suppuration.

The adhesive sealant of the present invention could improve the effectiveness of vitrectomy surgeries for retinal reattachment by providing short term bonding between the retina and retinal pigment epithelial (RPE) during the period in which laser-induced scars are forming. More broadly, the adhesive may provide a simple, safe, and effective alternative to existing soft-tissue adhesive.

The novel adhesive sealant of the present invention may optionally be used to connect an implant material to the tissue. Such an implant can be a neuronal tube guide. A neuronal tube can facilitates angiogenesis to improve neuronal regeneration. The described implant can have two layers. An inner tube made from a semipermeable gelatin foil represents the guiding compartment for regenerting axons and prevents infiltrtion from scar forming fibroblasts. A proangiogenic gelatin sponge layer around the inner tube is designed for enhanced blood vessel formation. The tube can be prepared by using chemical or enzymatic crosslinking in-vitro and thereafter it is preferably affixed in-situ using the adhesive sealant of the present invention.

According to some embodiments of the present invention, there is provided a matrix comprising at least one cross-linked gelatin layer, in which one or more substances are physically suspended within the cross-linked gelatin layer. Such a matrix may optionally be used for delivery of a therapeutic substance, including but not limited to any type of drug, protein, peptide, antibody, nucleotide based agent (such as DNA or RNA for example) and so forth.

Yet another embodiment of the present invention is to prevent post surgical adhesions. As described in the background, there is no good product for prevention of such adhesions. Omrix, Inc, Ness Ziona, Israel is developing a fibrin based anti-adhesion product. The present invention provides a protein based adhesive which can be crosslinked by any non-toxic crosslinker (Le., oxidative enzyme or transglutaminase), which as noted above is preferably an enzyme-crosslinked non-fibrin sealant, more preferably comprising gelatin. When applied on to the tissue and mixed with the crosslinker, the sealant completely polymerizes in-situ after about 3-5 minutes and thereafter will become a protective layer of hydrogel, which is incapable of further adherence. This layer acts as a barrier for the formation of new scar tissue. Such protective layer can prevents post surgical adhesion. In Example 4 it was demonstrated that the sealant of the present invention can adhere to a living abdominal tissue such as an intestine of pig. After about 3-5 minutes, polymerization is complete and the sealant will not further adhere to tissue. This suggests its use as post surgical barrier for adhesions. Optionally and preferably, the cross-linkable protein of the sealant of the present invention includes gelatin and any gelatin variant.

According to any of the embodiments of the present invention, gelatin may optionally comprise any type of gelatin which comprises protein that is known in the art, preferably including but not limited to gelatin obtained by partial hydrolysis of animal tissue and/or collagen obtained from animal tissue, including but not limited to animal skin, connective tissue (including but not limited to ligaments, cartilage and the like), antlers or horns and the like, and/or bones, and/or fish scales and/or bones or other components; and/or a recombinant gelatin produced using bacterial, yeast, animal, insect, or plant systems or any type of cell culture.

Any of the embodiments of the sealant of the present invention may be practiced by one having ordinary skill in the art upon perusal of the description herein together with PCT Application No. PCT/US2007/25726, by the inventors of the present invention, which is hereby incorporated by reference as if fully set forth herein. Additional compositions useful for implementing the teachings of the present invention are disclosed in the co-filed PCT Applications entitled “IMPROVED CROSS-LINKED COMPOSITIONS” and “A METHOD FOR ENZYMATIC CROSS-LINKING OF A PROTEIN” by the present inventors.

According to preferred embodiments of the present invention, gelatin from animal origins preferably comprises gelatin from mammalian origins and more preferably comprises one or more of pork skins, pork and cattle bones, or split cattle hides, or any other pig or bovine source. More preferably, such gelatin comprises porcine gelatin since it has a lower rate of anaphylaxis. Gelatin from animal origins may optionally be of type A (Acid Treated) or of type B (Alkaline Treated), though it is preferably type A.

Optionally and preferably, the non-toxic cross-linking material comprises an enzyme. Such an enzyme can be oxidative enzymes (tyrosinase, laccase) or transglutaminase (TG).

Optionally and preferably, the non-toxic cross-linking material comprises transglutaminase, which may optionally comprise any type of calcium dependent or independent transglutaminase, which may for example optionally be a calcium-independent microbial transglutaminase (mTG).

According to some embodiments, the sealant of the present invention further comprises a buffer (such as phosphate buffered saline, or a non-phosphate buffer (including include acetate buffer (such as sodium acetate), citrate buffer (such as sodium citrate), succinate buffer, maleate buffer, tris(hydroxymethyl)methylamine (TRIS), 3-{[tris(hydroxymethyl)methyl]amino}propanesulfonic acid (TAPS), N,N-bis(2-hydroxyethyl)glycine (bicine), N-tris(hydroxymethyl)methylglycine (tricine), 2-{[tris(hydroxymethyl)methyl]amino}ethanesulfonic acid (TES), 3-(N-morpholino)propanesulfonic acid (MOPS), piperazine-N,N′-bis(2-ethanesulfonic acid) (PIPES), dimethylarsinic acid, N-(2-hydroxyethyl)piperazine-N′-(2-ethane sulfonic acid) (HEPES), and 2-(N-morpholino)ethanesulfonic acid (MES)).

According to some embodiments, the sealant of the present invention further comprises at least one agent for lowering the cross-linking transition point of the cross-linkable protein or polypeptide, such as, for example, urea or calcium.

According to some embodiments, the sealant of the present invention further comprises a calcium sequestering agent, for example, polyphosphate salts, such as pyrophosphates (including tetrasodium pyrophosphate, disodium dihydrogen pyrophosphate, tetrapotassium pyrophosphate, dipotassium dihydrogen pyrophosphate, and dipotassium disodium pyrophosphate), tripolyphosphates (including pentasodium tripolyphosphate, and pentapotassium tripolyphosphate), higher polyphosphate salts such as sodium and potassium tetraphosphates, and hexametaphosphate salts, also known as ‘glassy phosphates’ or ‘polypyrophosphates’, and carboxylates, (such as alkali metal citrate salts, alkali metal acetate, lactate, tartrate and malate salts, alkali metal salts of ethylenediaminetetraacetic acid (EDTA), and editronic acid).

According to some embodiments, the sealant of the present invention further comprises a urea sequestering or urea hydrolyzing agent, such as urease.

The sealant optionally comprises additional excipients, such as, for example, a plasticizer, (such as citric acid alkyl esters, glycerol esters, phthalic acid alkyl esters, sebacic acid alkyl esters, sucrose esters, sorbitan esters, acetylated monoglycerides, glycerols, fatty acid esters, glycols, propylene glycol, lauric acid, sucrose, Methyl citrate, acetyl triethyl citrate, glyceryl triacetate, poloxamers, alkyl aryl phosphates, diethyl phthalate, mono- and di-glycerides of edible fats or oils, tributyl citrate, dibutyl phthalate, dibutyl sebacate, polysorbate, polyethylene glycols 200 to 12,000, Carbowax polyethylene glycols, polyvinyl alcohol and mixtures thereof); a surfactant (such as polysorbate 20 (Tween™ 20), polyoxyethyleneglycol dodecyl ether (Brij™ 35), and polyoxyethylene-polyoxypropylene block copolymer (Pluronic™ F-68)); or a coloring agent.

The sealant optionally further comprises an ammonia scavenging, sequestering or binding agent.

An example of such an agent is disaccharide lactulose. Lactulose is a synthetic disaccharide that is not hydrolysed by intestinal enzymes. Lactulose inhibits bacterial ammonia production by acidifying the content of the bowel. It promotes growth of colonic flora. The growing biomass uses ammonia and nitrogen from amino acids to synthesise bacterial protein, which in turn inhibits protein degradation to NH₃. Lactulose leads to less ammonia by inhibiting bacterial urea degradation and reduces colonic transit time, thus reducing the time available for ammonia production and expediting ammonia elimination. (Deglin J H, et al. Lactulose. In Davis's drug guide for nurses (9th ed., 2003) (pp. 589-590). Philadelphia: F. A. Davis.) Lactulose is commercially available from Solvay SA (Brussels), among other suppliers.

Another embodiment of this invention includes the use of a mixture of four forms of the strong cation exchange resin, Amberlite™ IR-120 (Advanced Biosciences, Philadelphia, Pa.), in the treatment of ammonia intoxication. This resin mixture, with a total quantity of 750 mEq, when used in the extracorporeal circulation system, was found to be efficient in the correction of hyperammonemia of experimental dogs and to be unaccompanied by any untoward effects. (Juggi J S, et al. In-Vivo Studies with a Cation Exchange Resin Mixture in the Removal of Excessive Ammonium from the Extracorporeal Circulation System. ANZ J Surg 1968; 38 (2): p 194-201).

Another embodiment of this invention includes the use of saponins, particularly yucca saponin, or the glyco-fraction derivative of Yucca shidigera plant, both of which have demonstrated ammonia-binding ability (Hussain I, Ismail A M, Cheeke P R. Animal Feed Science and Technology, 1996; 62 (2), p. 121-129).

Another embodiment of this invention includes the use of a sodium phenylacetate and sodium benzoate solution as an ammonia scavenger. Such a solution is commercially available under the trade name AMMONUL® (Medicis, Scottsdale, Ariz.), which consists of a solution of 10% sodium phenylacetate, 10% sodium benzoate.

In an alternate embodiment of the current invention, L-glutamine (L-Gln) or L-glutamate (L-Glu) is added to the protein-crosslinker composition, preferably to the protein component of the composition. L-Gln and L-Glu stimulate the metabolism of ammonia to urea in cells, and also inhibit the uptake and facilitates the extrusion of ammonia from cells (Nakamura E, Hagen S J. Am J of Phys. GI and Liver Phys, 2002; 46(6), p. G1264-G1275.). In an in situ cross-linking process that releases ammonia, L-Gln and/or L-Glu have utility in neutralizing the released ammonia by reducing the amount of ammonia absorbed by cells and by accelerating the cells' natural ability to metabolize ammonia.

According to any of the embodiments of the present invention, the biocompatible medical sealant is provided in the form of liquid, gel, spray, foam, or lyophilized form.

According to some embodiments, a mechanical supportive bio-adsorbable backing for the sealant is provided.

As illustrated in Example 5 below, the convergence of the sealant and the backing surprisingly augments the efficacy of the clinical outcome.

Optionally, the sealant may be dried together with the backing.

The components of the sealant preferably only begin crosslinking to create the curing effect of the sealant once they are applied together onto the tissue or at the tissue site where sealing is required or desired.

The components of the sealant create intermolecular chemical bonds both with the other sealant molecules and with the collagen of the extra cellular matrix of the applied tissue.

According to some embodiments, the biocompatible medical sealant of the present invention may be used for repair of retinal detachment.

In a preferred embodiment of the current invention, the crosslinkable protein solution and crosslinking material solution form a sealant by being processed through a mixing unit to achieve homogeneity of at least 95% immediately before coming into contact with the target biological system.

Preferably, the crosslinking material solution and crosslinkable protein solution achieve homogeneity of at least 98% after being process through a mixing unit.

The mixing unit for use with this embodiment can include dynamic mixing elements, static mixing elements, or a combination of the two. The mixing unit preferably mixes the material in a continuous process as the material is being applied, rather than preparing the entire batch of material at once and then applying it after mixing is completed for the entire batch.

Preferably, static mixing elements are used and the protein solution and crosslinking material solution are introduced to the static mixing unit at a volumetric ratio ranging from 10:1 to 1:10 crosslinking material solution to protein solution. More preferably, the volumetric ratio is 4:1 to 1:4.

In some embodiments of the current invention, the viscosity ratio between the protein solution and crosslinking material solution is greater than 10:1, preferably is greater than 50:1, and more preferably greater than 100:1.

In such embodiments, it was found that the most commonly used static mixer geometries, helical static mixers, were ineffective for mixing the protein solution and crosslinking material to homogeneity of above 95%. Such mixing elements are sold by many companies under a variety of brand names including Spiral Mixer™ (TAH Industries; Robbinsville, N.J.) and STATOMIX™ (ConProTec Inc; Salem, N.H.). Though both solutions were simultaneously introduced to the mixing units, the less viscous solution progressed more rapidly through the unit resulting in uneven mixture and early release of crosslinking material solution that had not been mixed with protein solution. This effect is known as fluid streaking.

PCT No. WO/2004/004875 and U.S. Pat. No. 6,773,156, both of which are hereby incorporated by reference as if fully disclosed herein, disclose an apparatus for reducing fluid streaking in a motionless mixer. The Turbo Mixer™ (TAH Industries; Robbinsville, N.J.) line of static mixer units is based on that invention. The current inventors have found the Turbo Mixer geometry surprisingly useful for mixing elements that are capable of mixing a protein solution and crosslinking material solution to homogeneity of above 95% when the initial viscosity ratio between the protein solution and crosslinking material solution is greater than 10:1. More surprisingly, it has been found that a Turbo Mixer static mixer can be used to mix these solutions to a homogeneity above 95% when the initial viscosity ratio is greater than 100:1.

Example 10 describes the use of the Turbo 295-620 mixer to form a sealant by mixing a gelatin solution (viscosity of approximately 5,000 cP) with a mTG solution (viscosity of approximately 20 cP). The mixed sealant is capable of sealing a simulated intestinal wound to 60 mmHg, demonstrating homogenous mixture of the two solutions.

In a preferred embodiment of mixing a protein solution with a crosslinking material solution where the viscosity ratio is greater than 10:1, the methods or apparatus for reducing fluid streaking in a motionless mixer of U.S. Pat. No. 6,773,156 are utilized to mix the solutions to a homogeneity of greater than 95%.

Preferably, more than 10 mixing elements are used and more preferably 20 or more mixing elements are used.

In another embodiment of mixing a protein solution with a crosslinking material solution where the viscosity ratio is greater than 10:1, a different static mixer unit is used that includes a mechanism of flow inversion, such as a flow inversion baffle, in order to maintain a homogeneous mixed composition.

### EXAMPLES

Reference is now made to the following examples, which together with the above description, illustrate the invention in a non limiting fashion.

### Example 1

### Lung Volume Reduction in a Rat Model

This Example provides an in vivo demonstration of a biocompatible medical sealant composition according to the present invention for achieving lung volume reduction. As described above, lung volume reduction has many therapeutic applications, particularly for diseases or conditions in which lung tissue becomes chronically distended, such as emphysema for example.

Young-adult male Sprague Dawley (SD) Rats were used.

Material: A medical sealant according to some embodiments of the present invention, featuring a gelatin component and an enzyme component, was used. The gelatin component featured 25% (w/w) gelatin (porcine, type A, 275 bloom) dissolved in a 0.1M Na—Ac (Sigma-Aldrich, St. Louis) buffer pH 6.0 at 37° C. with 3.8 M urea (Sigma-Aldrich, St. Louis) and 0.15M CaCl2 (Sigma-Aldrich, St. Louis). The enzyme component included 90 EU/mL of food grade microbial Transglutaminase enzyme (Activain WM, Ajinomoto™) maltodextrin dissolved in 0.2 M Na-Citrate (Sigma-Aldrich, St. Louis) buffer pH 6.0. Components were mixed in 2:1 gelatin to enzyme component volume ratio immediately prior to use, to initiate curing of the sealant.

Procedure: The animal was anesthetized and a minimal midline incision was made in its neck. A tracheotomy was performed and 18 G IV cannula (ø=1.3 mm, L=45 mm) was inserted to the animal's trachea.

0.25 mL of the sealant was injected slowly through the cannula using a 2.5 mL syringe. The cannula was extracted 1 minute after the material was applied. The neck incision was closed using metal staples and the animal was allowed to recover.

Animals were managed routinely until 15 days after the procedure. Observations included body weight and behavioral changes.

On day 15 the animals were sacrificed and their lungs were examined. Macroscopic evaluation was performed for anatomical changes and the lungs were sent for histology.

Results: Macroscopic evaluation as well as body weight measurements indicated reduced function of the lungs at the local site of the sealant implantation.

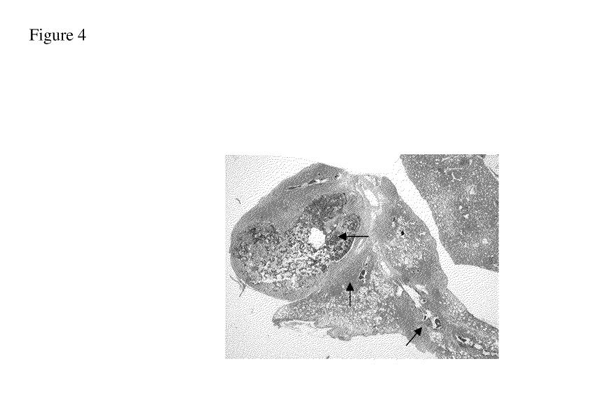

Histopathological evaluation performed by expert revealed remnants of the sealant in the animals' lungs (FIG. 4). The medical sealant partially obstructed the main lobes of the right lung of each animal, causing bronchitis obliterans along with subpleural fibrosis of the lung tissue. The sealant blocked the main stem bronchi of the right long lobe and inserted the lung bronchus, where it formed fibrosis of the lung's septa.

The results show that the medical sealant was able to obstruct the main bronchi of the lobes of the right lung. These results indicate that the medical sealant can serve as a lung volume reduction agent to be utilized to treat emphysema.

### Example 2

### Prevention of Post Surgical Adhesions in a Rat Model

This Example provides an in vivo demonstration of the successful use of a biocompatible medical sealant composition according to the present invention for the prevention of post surgical adhesions.

Young-adult male Sprague Dawley (SD) Rats were used.

Materials: A medical sealant according to some embodiments of the present invention, featuring a gelatin component and an enzyme component, was used. The gelatin component featured 25% (w/w) gelatin (porcine, type A, 275 bloom) dissolved in a 0.1M Na—Ac (Sigma-Aldrich, St. Louis) buffer pH 6.0 at 37° C. with 3.8 M urea (Sigma-Aldrich, St. Louis) and 0.15M CaCl2 (Sigma-Aldrich, St. Louis). The enzyme component included 90 EU/mL of food grade microbial transglutaminase enzyme (Activain WM, Ajinomoto™) maltodextrin dissolved in 0.2 M Na-Citrate (Sigma-Aldrich, St. Louis) buffer pH 6.0. Components were mixed in 2:1 gelatin to enzyme component volume ratio immediately prior to use, to initiate curing of the sealant.

Procedure: Each animal was anesthetized and the surgical site was depilated and disinfected using 70% ethanol solution. A midline laparotomy was performed. A 2 cm by 1 cm defect in the right abdominal wall, just above the cecum, was created by abrading for 10-15 strokes with moderate pressure using a #11 surgical blade. The cecum was exposed and abraded by scraping with a scalpel until a homogenous surface of petechial haemorrhages was formed over a 1×2-cm area. Access blood or tissue was removed using cotton swabs and gauze pads .

The cecum and abdominal defect were dried by exposure to air for 10 minutes. Other areas of the abdominal wall and the cecum were protected from drying by placing moist gauze over them during this period.

In the test group, 0.3 mL of a sealant was applied on top of the abraded cecum, using a 1 mL syringe. The sealant was dripped and then spread using a flexible cannula, to form a uniform layer.

In the control group, no material was applied.

In both the control and test group the cecum and abdominal wall were left exposed to air for another 10 minutes post application.

Upon closure, the cecum was positioned in such a way that it would contact the abdominal-wall defect. The abdominal wall was closed with a continuous nylon loop and the skin with metal staples.

14 days after surgery, the animals were euthanized. The skin and muscle layers of the abdomens were incised lateral and distal to the location of the original defect and the formation of adhesions was examined macroscopically. Tissue sections were sent for histology.

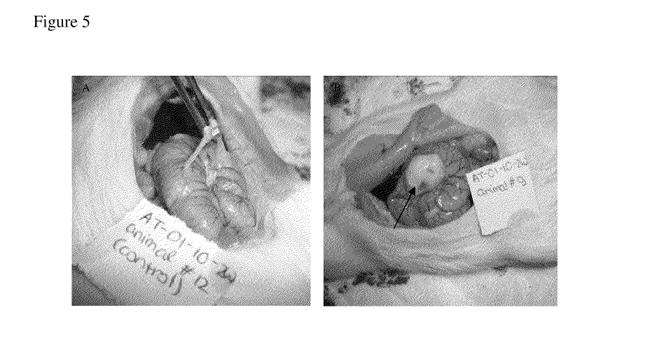

Results: Macroscopic evaluation showed distinctive differences between the control and test groups (FIG. 5). Accordingly, animals in which a medical sealant was used to cover the abrasion did not show any evidence for adhesion development (FIG. 5B shows a photograph of results from an exemplary animal in the test group). Nevertheless, all animals of the control, non-treated group developed moderate adhesions (FIG. 5A shows a photograph of results from an exemplary animal in the control) group. Histopathological examination of sections of the control and test groups supports these findings.

These results show that the application of a medical sealant prevents bowel adhesion formation.

### Example 3

### Dura Reconstruction

This Example provides an in vivo demonstration of the successful use of a biocompatible medical sealant composition according to the present invention for the reconstruction of dura and prevention of cerebro-spinal fluid leakage. The ability of the medical sealant in adhering to the dura tissue was examined in an acute model performed in a young LW swine.

Materials: a medical sealant according to some embodiments of the present invention, featuring a gelatin component and an enzyme component, was used. The gelatin component featured 25% (w/w) gelatin (porcine, type A, 275 bloom) dissolved in a 0.1M Na—Ac (Sigma-Aldrich, St. Louis) buffer pH 6.0 at 37° C. with 3.8 M urea (Sigma-Aldrich, St. Louis) and 0.15M CaCl2 (Sigma-Aldrich, St. Louis). The enzyme component included 90 EU/mL of food grade microbial Transglutaminase enzyme (Activain WM, Ajinomoto™) maltodextrin dissolved in 0.2 M Na-Citrate (Sigma-Aldrich, St. Louis) buffer pH 6.0. Components were mixed in 2:1 gelatin to enzyme component volume ratio immediately prior to use, to initiate curing of the sealant .

Procedure: The procedure was performed on a euthanized swine. Upon sacrifice, craniectomy was performed to reveal the animal's dura. An approximately 5 cm longitudinal dural incision was done. 1 mL of the sealant was applied to the incision site using a 5 mL syringe and left to cure for 3 minutes. The tissue was then excised and examined ex-vivo. Manual force was applied by the surgeon in order to examine the adherence of the sealant to the tissue.

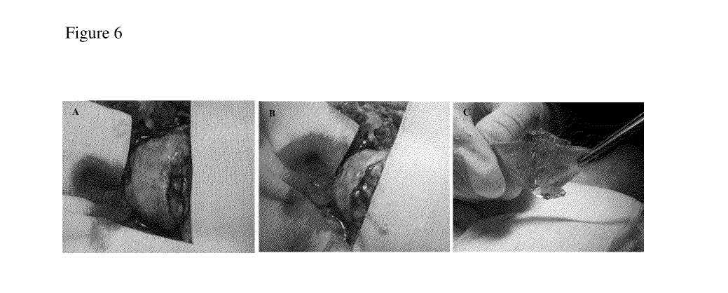

Furthermore, a piece of the dura tissue was excised and a longitudinal incision was made ex-vivo to separate two parts of the dura (see FIG. 6C). The medical sealant was used to connect between the two separated dura parts. 1 mL of the medical sealant was applied to connect the two dura parts and left to cure for 3 minutes. After 3 minutes the surgeon laterally pulled the dura parts and examined the stability of the sealant.

Results: The medical sealant strongly adhered to the dura tissue (see FIG. 6). The sealant formed a strong and uniform biomimetic film and the surgeon had to apply force in order to disconnect between the two glued dura parts.

These results show that the medical sealant adheres strongly to the dura tissue of a swine and can serve as an agent for dura reconstruction and prevention of cerebro-spinal fluid leakage.

### Example 4

### Reinforcement of Anastomosis Surgical Repair Lines

This Example provides an in vivo demonstration of the use of a biocompatible medical sealant composition according to the present invention, for the securing of surgical repair lines against leaks.

A deliberately perforated anastomosis was formed in the rectum of a young LW swine using a surgical stapler. The medical sealant according to the present invention was applied onto the perforation and examined for its ability to prevent leakage in an acute model and for its effect on the tissue reaction in a chronic model.

Materials: a medical sealant according to some embodiments of the present invention was used, featuring a gelatin component and an enzyme component. The gelatin component featured 25% (w/w) gelatin (porcine, type A, 275 bloom) dissolved in a 0.1M Na—Ac (Sigma-Aldrich, St. Louis) buffer pH 6.0 at 37° C. with 3.8 M urea (Sigma-Aldrich, St. Louis) and 0.15M CaCl2 (Sigma-Aldrich, St. Louis). The enzyme component included 90 EU/mL of food grade microbial Transglutaminase enzyme (Activain WM, Ajinomoto™) maltodextrin dissolved in 0.2 M Na-Citrate (Sigma-Aldrich, St. Louis) buffer pH 6.0. Components were mixed in 2:1 gelatin to enzyme component volume ratio immediately prior to use, to initiate curing of the sealant.

Procedure: Acute and chronic feasibility and safety models were implemented in a swine model. 12 hours pre-operation the animals were treated with laxatives and enema. The animals were anesthetized and a lower midline laparotomy was performed. The rectum was exposed. Two (2) adjacent staples were removed from a circular stapler containing 26 staples (PPC-EEA 28, Covidien, USA) to form a perforation. The perforation size was 6.75 mm in diameter. An anastomosis was performed in the proximal intra-peritoneal rectum of each animal by inserting the circular stapler trans-anally, ligating the tissue around the Anvil's shaft with a silk suture and firing the staples.

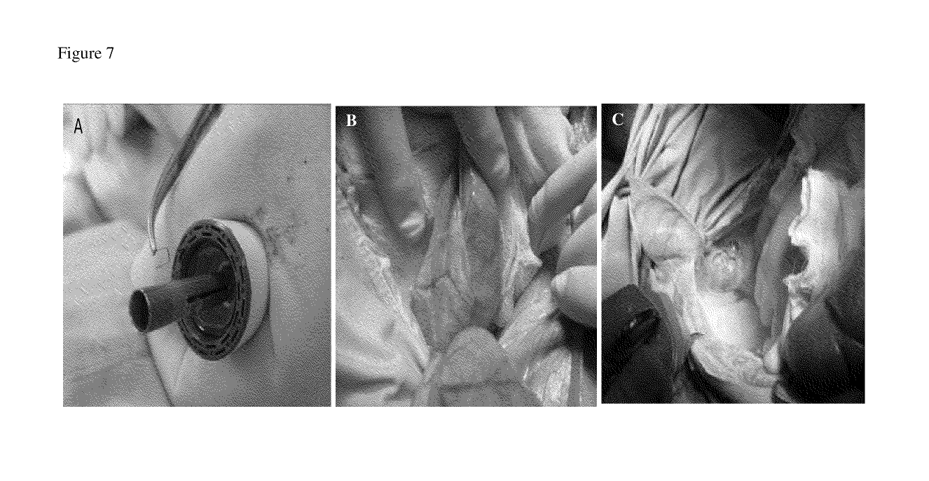

In the acute model, the ability of the sealant to prevent gastro-intestinal anastomosis leakage was examined. The abdominal space of the animal was filled with saline and air was insufflated through the animal's anus to determine the baseline leakage. Leakage is defined as air or liquid leakage in a pressure of 30-40 PSI and can be determined by the formation of air bubbles (see FIG. 7). Pressure was monitored using a manometer. After examining the baseline leakage the saline was removed and 5 mL of the sealant was applied on the deliberately perforated anastomosis using a 5 mL syringe. The sealant was left to cure for 4 minutes. Air was pumped with increasing pressure using the manual air pump and the burst pressure was determined by the appearance of air bubbles. The procedure was repeated in the lower and the upper rectum.

In the chronic model, the safety of the sealant was demonstrated. The animal was anesthetized and a lower midline laparotomy was performed using a number 20 surgical blade. The proximal intraperitoneal rectum was exposed. Two (2) adjacent staples were removed from a circular stapler containing 26 staples (PPC-EEA 28, Covidien, USA) to form a perforation. The perforation size was 6.75 mm in diameter. The anastomosis was performed in the proximal intra-peritoneal rectum by inserting the circular stapler trans-anally, ligating the tissue around the anvil's shaft with a silk suture and firing the staples. 5 mL of the sealant was applied on the entire circumference of the external surface of the anastomosis staple line, to prevent leakage from the deliberately perforated anastomosis and secure the anastomotic line. The sealant was left to cure for 6 minutes. The abdominal cavity was closed with continuous nylon loop for the fascia and continuous 2/0 vicryl for the sub-coetaneous fascia and metal staples for the skin. On postoperative day 7 the animal was operated under general anaesthesia. A second laparotomy was performed and the anastomosis was revealed. The anastomosis was excised and sent for histology.

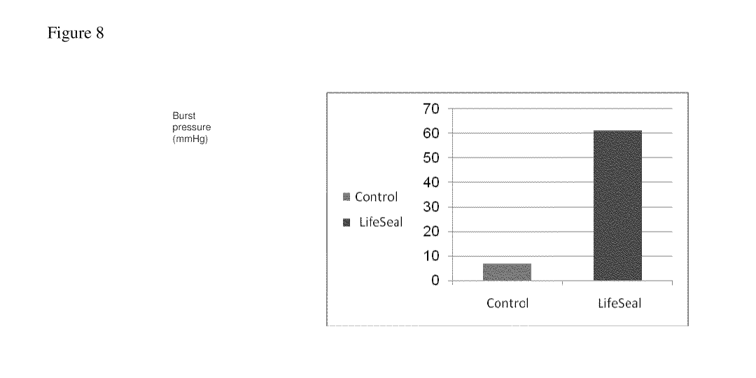

Results: Summary of the results of the acute model can be found in FIG. 8. Tissue sealed with the the sealant successfully withstood pressure as high as 70 mmHg. The average burst pressure was 61 mmHg.

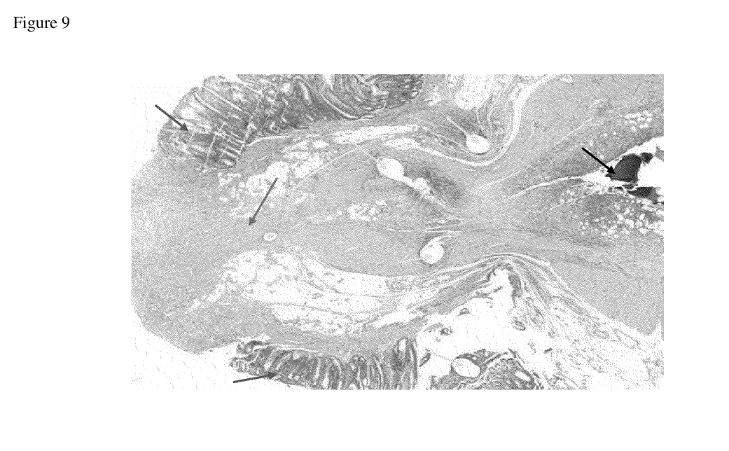

In the chronic model, no signs of adhesions or leakage were found. Histopathological evaluation showed that the sealant was well tolerated. The sealant material did not interfere with the natural healing process. The characteristic inflammatory reaction to the sealant material was a typical mild foreign body granulation reaction and a well circumscribed capsule was formed (results are shown in FIG. 9).

These results show that the medical sealant can successfully secure an anastomosis staple line. The sealant successfully prevented leakage from a perforated anastomosis in a swine's rectum. The sealant successfully withstood pressure as high as 70 mmHg while maintaining the seal.

As indicated by the acute model, the tissue reaction to the sealant is characterized by a capsular reaction. The capsule reinforces the anastomosis staple line and helps to prevent leakage formation; thus its formation is desirable, yet is not necessarily found after application of other sealants, other than those of the present invention. The sealant successfully prevented leakage from a perforation performed in a swine's anastomosis.

### Example 5

### Attachment of Implant Material to Tissue

This example provides an in vivo demonstration of the use of a biocompatible medical sealant composition according to the present invention, for the attachment of implant material to tissue.

Multiple size incisions were performed in the aorta or vena cava of a young LW swine. The medical sealant was applied in conjugation to a collagen or cellulose backing to seal the incision and prevent bleeding.

Materials: a medical sealant according to some embodiments of the present invention, comprising a gelatin component and an enzyme component was used. The gelatin component featured 25% (w/w) gelatin (porcine, type A, 275 bloom) dissolved in a 0.1M Na—Ac (Sigma-Aldrich, St. Louis) buffer pH 6.0 at 37° C. with 3.8 M urea (Sigma-Aldrich, St. Louis) and 0.15M CaCl2 (Sigma-Aldrich, St. Louis). The enzyme component included 90 EU/mL of food grade microbial Transglutaminase enzyme (Activain WM, Ajinomoto™) maltodextrin dissolved in 0.2 M Na-Citrate (Sigma-Aldrich, St. Louis) buffer pH 6.0. Components were mixed in 2:1 gelatin to enzyme component volume ratio immediately prior to use, to initiate curing of the sealant.

Procedure: The ability of the sealant to attach a material implant to vascular tissue was examined in active and non-active bleeding models.

In the active bleeding model the animal was anesthetized and IV administered with 5000 Units of Heparin to reduce its coagulation and 4 mg of Adrenalin to boost its blood pressure. A 5 mm incision was performed in the vena cava using a number 11 surgical blade, to cause severe bleeding. 3 mL of the medical sealant was applied in conjugation with a cellulose pad backing and manually pressed against the actively bleeding injury. After 4 minutes the compression was relieved and hemostasis was examined. The wound site was reexamined one hour later. The experiment was repeated with the use of cellulose backing alone, as a Control.

In the non-active bleeding models the animal was anesthetized and a 4 mm incision was performed in the aorta of a heparinized swine (5000 units, IV). The artery was clamped from both sides and 3 mL of the sealant to the incision site with a cellulose backing. After 4 minutes, the clamps were removed to allow blood flow renewal and the adherence of the implant was examined. The animal's body was physically agitated to examine the sealant's durability and the sealed wound site was examined for 1 hour and then the animal was administered with adrenalin (4 mg, IV).

In another non-active bleeding model a 3.3 mm punch was performed in the animal's aorta using a biopsy punch. The artery was clamped from both sides to prevent bleeding. 3 mL of the sealant was applied in conjugation to a collagen backing to the punctured aorta and left to cure. After 4 minute the clamps were removed and the bleeding and adherence of the implant were examined.

Results: In both the active and non-active bleeding models, the sealant successfully adhered to the vascular tissue and attached the implant thereto while preventing bleeding.

In the active bleeding model, the sealant stopped severe active bleeding from a vena cava injury performed in a swine administered with heparin. The cellulose implant remained attached to the wound site for more than 1 hour and after increasing the blood pressure using adrenalin. In comparison, the control cellulose backing failed to adhere to the bleeding tissue.

In the non-active bleeding models the sealant successfully attached an implant to the aorta. In the 4 mm incision in the heparinized swine sealant prevented bleeding and the implant remained attached to the tissue for the entire hour in which it was monitored. The sealant also successfully attached a collagen implant to a 3.3 mm diameter punch in the aorta and prevented the wound from bleeding.

The medical sealant successfully attached implant material, namely collagen and cellulose backings, to blood vessels that were actively and non-actively bleeding. The sealant remained durable for more than 1 hour, and was able to prevent bleeding even at both moderate and severe bleeding pressure.

### Example 6

### Vascular Sealing and Hemostasis

This example provides an in vivo demonstration of the use of a biocompatible medical sealant composition according to the present invention and its ability to stop or seal moderate or severe vascular bleeding.

Multiple sizes of incisions were performed in the different arteries of a young LW swine. The medical sealant was applied to the wound site in both active and non-active bleeding models and its ability to stop bleeding and seal the blood vessels was examined.

Materials: The medical sealant according to some embodiments of the present invention, comprising a gelatin component and an enzyme component, was used. The gelatin component featured 25% (w/w) gelatin (porcine, type A, 275 bloom) dissolved in a 0.1M Na—Ac (Sigma-Aldrich, St. Louis) buffer pH 6.0 at 37° C. with 3.8 M urea (Sigma-Aldrich, St. Louis) and 0.15M CaCl2 (Sigma-Aldrich, St. Louis). The enzyme component included 90 EU/mL of food grade microbial Transglutaminase enzyme (Activain WM, Ajinomoto™) maltodextrin dissolved in 0.2 M Na-Citrate (Sigma-Aldrich, St. Louis) buffer pH 6.0. Components were mixed in 2:1 gelatin to enzyme component volume ratio immediately prior to use, to initiate curing of the sealant.

Procedure: The ability of the medical sealant, in sealing and preventing bleeding from major arteries, was examined in the femoral and carotid arteries. The animals were anesthetized and the arteries were exposed.

A 2-3 mm incision was made using a number 11 surgical blade in a heparinised swine (10,000 Units, IV). The artery was clamped from both sides and 3 mL of the medical sealant was applied to the incision and left to cure. After 4 minutes the clamps were removed and hemostasis was examined. The wound site was agitated and the blood flow was examined using a Doppler meter.

In the femoral artery model, a 2-3mm incision was performed in the femoral artery of a swine administered with adrenalin (8 mg). The artery was clamped from both sides and 3 mL of the medical sealant was applied to the incision and left to cure. After 4 minutes the clamps were removed and hemostasis was examined. The wound site was agitated and the blood flow was examined using a Doppler meter.

Results: the sealant sealed and prevented bleeding from all of the 2-3 mm incisions made in the femoral and carotid arteries in swine administered with Heparin or Adrenalin. The sealant was durable after agitating the wound site. In all arterial bleeding experiments, healthy blood flow continued in the injured artery after the sealant had closed the wound.

The sealant successfully stopped moderate and severe bleeding from bleeding wounds in major arteries.

### Example 7

### Sealing Lung Perforations in a Swine Model

This example provides an in vivo demonstration of a biocompatible medical sealant composition according to the present invention for sealing lung perforations.

The parenchyma tissue of LW swine was injured and the ability of a medical sealant to prevent leakage was examined in an acute model.

Material: The medical sealant according to some embodiments of the present invention, comprising a gelatin component and an enzyme component, was used. The gelatin component featured 25% (w/w) gelatin (porcine, type A, 275 bloom) dissolved in a 0.1M Na—Ac (Sigma-Aldrich, St. Louis) buffer pH 6.0 at 37° C. with 3.8 M urea (Sigma-Aldrich, St. Louis) and 0.15M CaCl2 (Sigma-Aldrich, St. Louis). The enzyme component included 90 EU/mL of food grade microbial Transglutaminase enzyme (Activain WM, Ajinomoto™) maltodextrin dissolved in 0.2 M Na-Citrate (Sigma-Aldrich, St. Louis) buffer pH 6.0. Components were mixed in 2:1 gelatin to enzyme component volume ratio immediately prior to use, to initiate curing of the sealant.

Procedure: During the following procedures the animal was maintained on general anaesthesia. The animal was placed in a supine position and artificially ventilated (tidal volume=300 mL). The right lung was exposed. 6 mm long and 2.5 cm deep incisions were made using a number 11 blade at the lung parenchyma. Ventilation was either halted for 1-2 minutes or was not disturbed and 1.5 mL of the sealant was immediately applied on top of the wound site using a syringe. After 1-2 minutes the ventilation was restored (tidal volume=400 mL, 15 inhalations per minute) and after another 1.5 minutes the animal's thorax was filled with warm saline solution and the formation of air bubbles due to air leakage through the perforated lung was examined. In one experiment the tidal volume was gradually increased until it reached 600 mL and the wound was physically agitated to examine the robustness of the sealing.

Results: the medical sealant adhered to the lung parenchyma and prevented air or blood leakage from 6 mm long and 2.5 cm deep incisions. The bond provided by the sealant was durable after increasing the tidal volume to 600 mL and the perforation was maintained in the sealed state. Examination of the sealant's effect after 3 hours showed that the sealant's bond remained durable and adhered strongly to the lung tissue. Agitation of the wound site did not affect the sealing durability.

The results show that the medical sealant successfully sealed a 6 mm long and 2.5 cm deep perforation in the lung parenchyma and that the bond provided thereto remained durable for at least 3 hours. This indicates that the medical sealant can serve as a lung sealing and lung reduction sealant.

### Example 8

### Effect of Sealant on Sealing of the Lymphatic System

A surgical procedure is performed on a patient, which encompasses removal of one or more lymph nodes. For example, the surgical procedure may optionally be breast cancer surgery that includes the removal of one or more lymph nodes. The sealant of the present invention, according to any of the above described embodiments, is applied to the body of the patient at the vicinity of the removed lymph node(s), thereby sealing the lymphatic system of the patient and preventing or at least reducing leakage of lymph from the area of node removal.

### Example 9

### Water Uptake of mTG Crosslinked Gelatin Gel

### Experimental Procedure

The main purpose of this study is to determine the amount of water uptake by the adhesive.

The sealant according to some embodiments of the present invention used in this study contained both microbial transglutaminase (mTG) and gelatin. The gelatin was 300 bloom, and was prepared as a 25% stock solution. The final concentration of gelatin in the adhesive was 17%. The mTG was prepared as a 20% stock solution from powder comprising 1% enzyme and 99% maltodextrin carrier, and the final activity in the adhesive was 40 U/g of gelatin (6.7 U/mL). After both the mTG and gelatin solutions were mixed, they were allowed to react in an incubator at 37° C. for 30 minutes. After reaction, the adhesive was cut into four samples, dried, and then weighed.

Experiment 1: After weighing, each sample was placed in a Petri dish containing 25 mL of PBS buffer and the Petri dishes were placed in an incubator at 37° C. Over the course of the 45 hour experiment, samples were intermittently removed from the incubator, touch dried and weighed as described above.
Experiment 2: As for experiment 1 except that the second was performed over 47 hours.

### Results

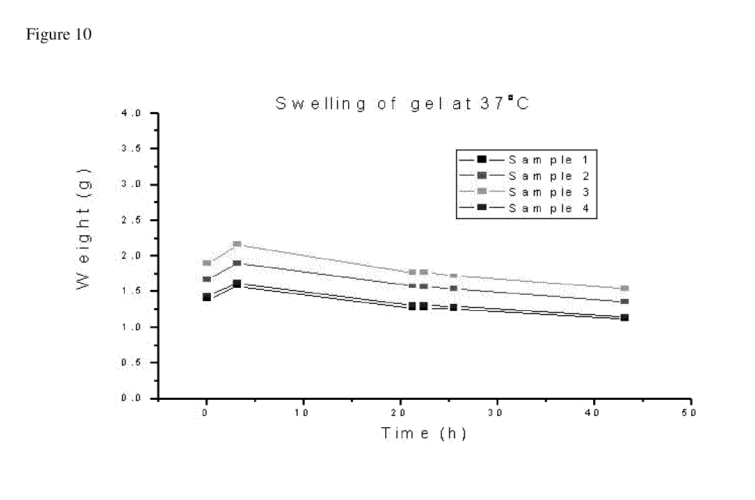

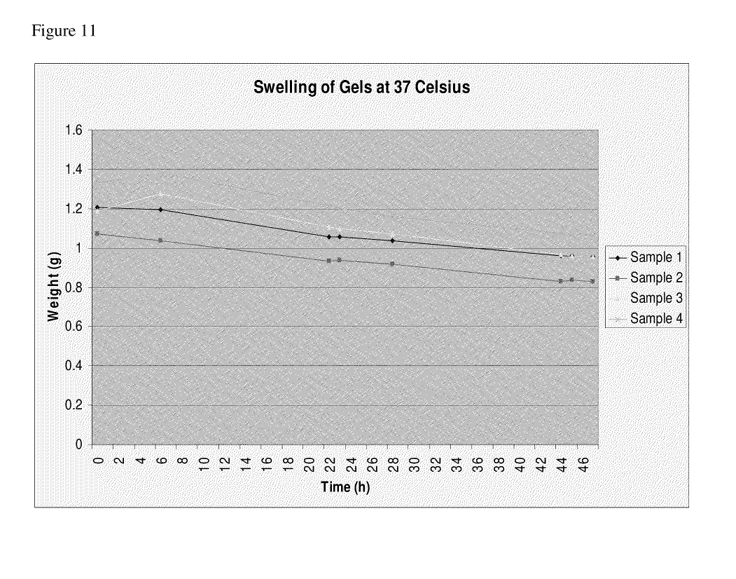

As shown in FIGS. 10 and 11, the samples show a small increase in weight for the gel over the first few hours. This apparent swelling may be attributed to the fact that the adhesive was prepared without being fully immersed in liquid and likely retains some swelling capacity. In both experiments, after this initial increase, no further increase in mass was observed during incubation. This indicates little, if any, swelling of the mTG crosslinked gelatin gel.

### Example 10

### Controlled Drug Delivery

This example provides an initial in vitro demonstration of a gel matrix made of an enzymatically crossed linked protein that serves as a drug delivery system with a controlled release.

Materials:

A gelatin component containing 25% (w/w) gelatin (porcine, type A, 275 bloom) dissolved in a 0.1M Na—Ac (Sigma-Aldrich, St. Louis) buffer pH 6.0 at 37° C. with 3.8 M urea (Sigma-Aldrich, St. Louis) and 0.15M CaCl2 (Sigma-Aldrich, St. Louis) was prepared. Food grade microbial Transglutaminase enzyme (Activa in maltodextrin WM) was obtained from Ajinomoto™. 0.2M Na-Citrate (Sigma-Aldrich, St. Louis) buffer pH 6.0 was prepared. Bromophenol Blue (Mw=691.9) was purchased from Bio-Rad Laboratories (CA). Dulbecco's Phosphate Buffered Saline (PBS) was obtained from Biological Industries (Kibbutz Beit HaEmek).

Procedure:

Enzyme components containing 30 EU/mL and 90 EU/mL were prepared by dissolving microbial transglutaminase in Na-Citrate buffer containing 0.5% (w/v) Bromophenol Blue. 0.66 mL of each enzyme component was mixed with 1.33 mL of the gelatin component to yield a crossed linked gel matrix. The mixed solutions were immediately cast to moulds to form triplicates of 1.7 mm thick films and left to cure for 9 minutes. The final Bromophenol blue concentration was 4.93 mM. After 9 minutes each film was submerged in 20 mL of PBS solution and incubated at 37° C. with orbital shaking. Bromophenol Blue concentrations in the extracts was measured at t=0, 1 h, 2 h and 24 h. The optical density of the extract solutions was measured using a spectrophotometer at the wavelength of 592 nm. The concentration was determined using the molar extinction coefficient of Bromophenol Blue (ε592 nm=78000).

To examine the effect of the cross linking density on the drug release kinetics from the gel matrix, the release from gels prepared with 30 EU/mL microbial transglutaminase components were compared to the kinetics of gels prepared with 60 EU/mL microbial transglutaminase components.

Results:

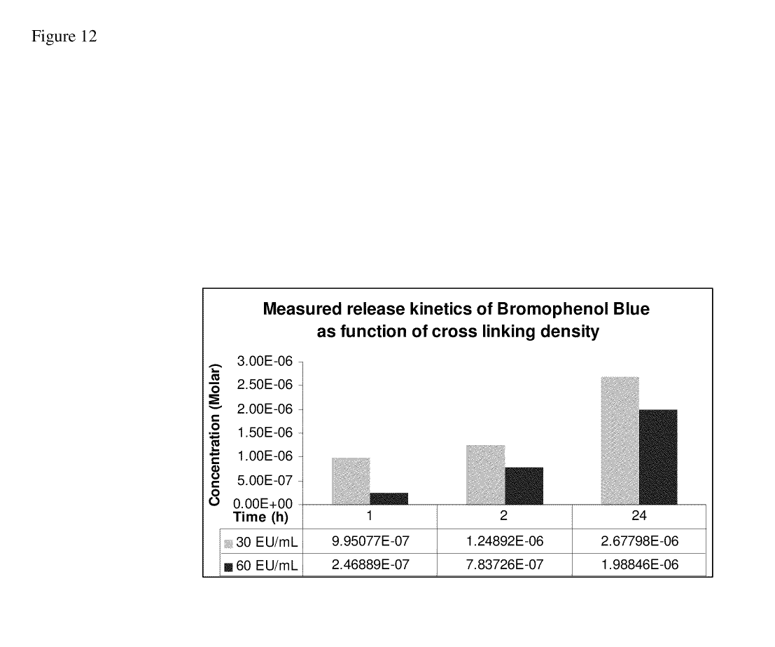

FIG. 12 shows the results for the Bromophenol Blue concentrations released from the gels as function of time. The blue bars shows the measured concentrations from gels crossed linked with enzyme component containing 30 EU/mL and the red bars indicate the concentrations released from gels crossed linked with enzyme component containing 60 EU/mL.

The results show that Bromophenol Blue is released to the matrix in a controlled manner over time, as indicated by the increase of its concentration in the extract solution as function of time.

The results also indicate that the amount of cross linker in the gel affects the release of Bromophenol Blue from the gel matrix. The higher the enzyme concentration is, the lower the released Bromophenol Blue concentration.

The results indicate that the suggested gel matrix can serve as a drug delivery system. Controlled release of small molecules such as Bromophenol Blue (Mw=691.9) is feasible. The release profile can be tailored through controlling the cross linking density of the gel matrix.

### Example 11

### Applicator for Sealant/Adherent

Example 11 describes the use of the Turbo 295-620 mixer to form a sealant by mixing a gelatin solution with a mTG solution. The mixed sealant is capable of sealing a simulated intestinal wound to approximately 60 mmHg, demonstrating homogenous mixture of the two solutions.

Materials

The following materials were used in the experiment: Gelita 275 bloom, type A porcine gelatin (Gelita, Sioux City), Urea 99.5% (Sigma-Aldrich, St. Louis), Calcium Chloride (Sigma, St. Louis, Mo.), Sodium Acetate trihydrate (Sigma-Aldrich, St. Louis), Acetic Acid 100% (Ridel-De Haen), Sodium Citrate (Sigma-Aldrich, St. Louis), Citric Acid Monohydrate (Sigma-Aldrich, St. Louis), ACTIVA™ TG microbial transglutaminase (mTG) product (10% protein, 90% maltodextrin—Ajinomoto, Japan).

Methods

The following solutions were prepared:

Gelatin solution: 25% (w/w) gelatin solution with 3.8M Urea, 0.15M CaCl2, 0.1M Sodium Acetate.

mTG solution: 7.5% (w/w) solution of ACTIVA TG in 0.2M Na-Citrate

2 mL aliquots of mTG solution were filled into 3 mL polycarbonate syringes (Merit Medical Systems; South Jordan, Utah). 4 mL aliquots of gelatin solution were filled into 6 mL polycarbonate syringes (Merit Medical Systems; South Jordan, Utah).

One mTG solution syringe and one gelatin solution syringe were connected to each of 3 Turbo 295-620 mixers (TAH; Robbinsville, N.J.) using a Y junction.

A burst pressure testing system was assembled that enabled a segment of explanted pig intestine to be pressurized using a hand-held air pump. A Y junction connected the air pump to a manometer (Digital Pressure Indicator, DPI 705 model, Druck Limited Mfg.) such that the manometer displayed the pressure in the intestine segment. A segment of the intestine, containing a single incision, is submerged in a water bath. The segment is clamped in both sides while a tube is inserted through it. The tube is connected to a manual air pump with a manometer.

Six explanted pig intestine segments were prepared. On each segment, one 3 mm incision was made. To three of the segments, the incision was then covered with a mixture of the gelatin and mTG solutions that was mixed at a 2:1 volumetric ratio (gelatin:mTG) through the mixer. These were known as the sealant group. Three of the segments were not treated with sealant.

The six segments were then loaded one by one into the burst pressure testing system and covered in saline. For each, the pressure was increased by introducing 20 mL of air per minute. When bubbles escaped through the incision site, this was considered the “burst pressure” for that sample. The results for the control group and Turbo mixer sealant group are described below.

Results

| | Burst Pressure at |
|---|---|
| | t = 0 (mmHg) |
| Control | 7 ± 2.8 |
| Sealant | 62 ± 21.7 |

These results indicate that the Turbo mixer was successful in mixing the gelatin and mTG solutions to form an efficacious sealant.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-61. (canceled)

62. A method of repairing a tissue of a subject in need of repair thereof, comprising applying to the tissue a biocompatible medical adherent composition comprising gelatin, microbial transglutaminase for inducing cross-linking of said gelatin, wherein the gelatin transition point of the gelatin solution has been reduced or the solubility of the gelatin has increased, for thereby adhering at least a portion of the biological tissue, for an application selected from the group consisting of reinforcement of surgical repair lines; preventing anastomic dehiscence; inducing pneumostasis; sealing lung tissue; sealing dura tissue; sealing around an insertion wound into the biological tissue made by insertion of an implanted medical device or due to withdrawal of said implanted medical device; attaching the biological tissue to an artificial material; closing a fistula; preventing adhesion of the biological tissue to another biological tissue; performing lung volume reduction; inducing lymphostasis after lymphorrhea; and sealing a cerebrospinal fluid leak.

63. The method of claim 62, wherein the composition further comprises a transition point lowering agent for lowering the gelatin transition point.

64. The method of claim 63, wherein said transition point lowering agent is selected from the group consisting of urea and calcium.

65. The method of claim 64, wherein said surgical repair lines comprise one or more of staple lines and suture lines.

66. The method of claim 64, wherein said medical device comprises a catheter.

67. The method of claim 64, wherein said medical device is a permanent device, and said sealing is performed around said device.

68. The method of claim 67, wherein said permanent device is a stoma tube.

69. The method of claim 64, wherein said lymphorrhea occurs after surgical lymph-node dissection.

70. The method of claim 69, wherein said surgical lymph node dissection is selected from the group consisting of auxiliary surgical lymph-node dissection, groin surgical lymph-node dissection, neck surgical lymph-node dissection, and pelvic and retroperitoneal surgical lymph-node dissection.

71. The method of claim 64, wherein said cerebro-spinal fluid leakage occurs due to a surgical procedure selected from the group consisting of brain surgery or injury and spinal surgery or injury.

72. The method of claim 64, wherein the sealant further comprises at least one additional protein or polypeptide.

73. The method of claim 64, wherein the sealant further comprises an antibiotic, an anesthetic or both.

74. The method of claim 64, wherein the sealant further comprises a material selected from the group consisting of a calcium sequestering agent, a urea sequestering agent, a urea hydrolyzing agent and ammonia scavenging agent.

75. The method of claim 64, wherein the sealant is applied in the form of a liquid, gel, spray, foam, or lyophilized form.

76. method of claim 64, wherein the sealant is attached to a supportive bio-absorbable backing and is applied with said backing.

77. The method of claim 76, wherein said sealant is dried together with said supportive bio-absorbable backing.

78. The method of claim 64, wherein said application comprises reducing lung volume in the subject, wherein said applying the composition comprises (a) collapsing a target region of the subject's lung; and (b) administering, by way of the subject's trachea, to the target region of the subject's lung: (i) a first composition comprising said gelatin and (ii) a second composition comprising said transglutaminase, whereafter one portion of the target region adheres to another portion of the target region, thereby reducing the subject's lung volume.
